(19) United States
(12) Patent Application Publication
(10) Pub. No.: US 2023/0012309 A1
(43) Pub. Date: Jan. 12, 2023

(54) GINGIVA STRIP PROCESSING USING ASYNCHRONOUS PROCESSING

(71) Applicant: Align Technology, Inc., San Jose, CA (US)

(72) Inventors: Andrey ROMANOV, Moscow (RU); Dmitry KIRSANOV, Moscow (RU)

(21) Appl. No.: 17/862,355

(22) Filed: Jul. 11, 2022

Related U.S. Application Data
(60) Provisional application No. 63/220,440, filed on Jul. 9, 2021.

(51) Int. Cl. A61C 7/00; A61C 7/08; A61C 9/00

(52) U.S. Cl. CPC A61C 7/002; A61C 7/08; A61C 9/0046

(57) ABSTRACT

Methods and apparatuses for asynchronously identifying and modeling a gingiva strip from the three-dimensional (3D) dental model of the patient's dentition. These methods may reduce the time required to generate accurate 3D dental models and therefore may reduce and streamline the process of generating dental treatment plans.

### CLAIM OF PRIORITY

This patent application claims priority to U.S. Provisional Application No. 63/220,440, titled “AUTOMATIC ATTACHMENT MATERIAL DETECTION AND REMOVAL,” filed on Jul. 9, 2021, and herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Orthodontic and dental treatments using a series of patient-removable appliances (e.g., “aligners”) are very useful for treating patients, and in particular for treating malocclusions. Treatment planning is typically performed in conjunction with the dental professional (e.g., dentist, orthodontist, dental technician, etc.), by generating a model of the patient's teeth in a final configuration and then breaking the treatment plan into a number of intermediate stages (steps) corresponding to incremental movements of the patient's teeth from an initial position towards the final position. Individual appliances are worn sequentially to move the teeth in each stage. This process may be interactive, adjusting the staging, and in some cases the final target position, based on constraints on the movement of the teeth and the dental professional's preferences. Once the final treatment plan is finalized, the series of aligners may be manufactured based on the treatment plan.

This treatment planning process may include many manual steps that are complex and may require a high level of knowledge of orthodontic norms. Further, because the steps are performed in series, the process may require a substantial amount of time. Manual steps may include preparation of the model for digital planning, reviewing and modifying proposed treatment plans (including staging) and aligner features placement (which includes features placed either on a tooth or on an aligner itself). These steps may be performed before providing an initial treatment plan to a dental professional, who may then modify the plan further and send it back for additional processing to adjust the treatment plan, repeating (iterating) this process until a final treatment plan is completed and then provided to the patient.

Sometimes a patient's teeth do not move according to the treatment plan. In such cases, the patient's teeth may be “off-track” and the treatment plan may be adjusted. A dental professional may re-scan the patient's teeth in order to develop a new treatment plan. The rescan may include artifacts, such as attachments that have been applied to a patient's teeth. In present systems, these artifacts may lead to less than desirable treatment plans.

The methods and apparatuses described herein may improve treatment planning, including potentially increasing the speed at which treatment plans may be completed, as well as providing greater choices and control to the dental professional.

### SUMMARY OF THE DISCLOSURE

As will be described in greater detail below, the present disclosure describes various apparatuses (e.g., systems, devices, etc., including software and one or more user interfaces) and methods for automatically detecting and removing attachment material from 3D model data and for guiding a user, such as a dentist, orthodontist or other dental professional, in removing one or more attachments.

Also described herein are methods and apparatuses for asynchronously processing portions of a process for modifying a 3D model of a patient's dentition, e.g., in parallel with one or more user-performed steps in order to streamline or reduced the time needed for processing the 3D model, and/or for generating a treatment plan. In general, these methods may include generating one or more copies of all or a portion of a 3D model of the patient's dentition, e.g., model data of a dental structure of a patient, which may be referred to equivalently herein as a digital model of a patient's dentition, and allowing the user to review and/or modify a proposed change to the original 3D model while concurrently and in parallel modifying the copy or copies of the 3D model according to the proposed change. For example, the methods and apparatuses described herein may improve the speed of removing one or more attachments (“white attachments”) and segment the teeth, and/or to identify and modify the 3D model to include a gingiva strip.

The systems and methods described herein may improve the functioning of a computing device by reducing computing resources and overhead for acquiring and storing updated patient data, thereby improving processing efficiency of the computing device over conventional approaches. These systems and methods may also improve the field of orthodontic treatment by analyzing data to efficiently target treatment areas and providing patients with access to more practitioners than conventionally available.

For example, described herein are method for identifying attachments in a digital model of a patient's teeth. The digital model of the patient's teeth may be referred to herein as three-dimensional (3D) dental model data. Thus, described herein are methods or procedures for identifying (e.g., automatically identifying) and/or for adjusting three-dimensional (3D) dental model data. These procedures may be used as part of any of the methods described herein, including as part of a method of guiding a user in removing one or more attachments.

For example, a method or part of a method may include: receiving model data of a dental structure of a patient (e.g., receiving a digital model including the patient's teeth), the model data including one or more attachments on the dental structure; detecting, from the model data, the one or more attachments on the dental structure. In some examples the method may also include modifying the model data to remove the detected one or more attachments and presenting the modified model data.

Detecting the one or more attachments may include: retrieving a previous model data of the dental structure; matching one or more teeth of the previous model data with respective one or more teeth of the model data; identifying one or more previous attachments from the previous model data; detecting one or more shape discrepancies from the model data; and identifying the one or more attachments from the model data using the one or more previous attachments and the one or more shape discrepancies.

Modifying the model data may further include, for each of the detected one or more attachments: calculating a depth from the attachment to a corresponding tooth based on the previous model data; and adjusting a surface of the attachment towards a direction inside the tooth based on the calculated depth. In some examples, adjusting the surface of the attachment may further include moving scan vertices inside a detected area corresponding to the attachment in the direction inside the tooth.

Presenting the modified model data may further comprise displaying visual indicators (e.g., color, arrows, circles, etc.) of the removed one or more attachments.

Detecting the one or more attachments may include: detecting, using a machine learning model, extra material on the dental structure; and identifying the extra material as the one or more attachments. For example, modifying the model data may further comprise, for each of the detected one or more attachments: predicting, using the machine learning model, a depth from the attachment to a corresponding tooth; and adjusting a surface of the attachment towards a direction inside the tooth based on the predicted depth.

In some examples detecting the one or more attachments further comprises: identifying a first set of potential attachments using previous model data; identifying a second set of potential attachments using a machine learning model; and identifying the one or more attachments based on cross-validating the first set of potential attachments with the second set of potential attachments. For example, identifying the one or more attachments based on cross-validating may further comprise discarding potential attachments of the first or second set of potential attachments that are close to interproximal or occlusal tooth areas if another of the first or second set of potential attachments lacks matching potential attachments.

Identifying the one or more attachments based on cross-validating may further comprise discarding, from the second set of potential attachments, potential attachments for areas that do not have significant deviations in the model data compared to the previous model data. In some examples identifying the one or more attachments based on cross-validating further comprises discarding, from the first set of potential attachments, potential attachments having a small distance to a corresponding tooth surface that do not intersect with potential attachments of the second set of potential attachments.

Presenting the modified model data may further comprise displaying, with corresponding confidence values, a plurality of attachment removal options based on the detected one or more attachments. The confidence values may be based on a degree of similarity between corresponding attachments detected via a plurality of detection approaches.

In some examples the methods may include updating a treatment plan for the patient using the modified model data. In some examples the method may include fabricating an orthodontic appliance (or a series of orthodontic appliances, e.g., aligners) based on the treatment plan.

Also described herein are methods including: receiving model data of a dental structure of a patient; retrieving previous model data of the patient; matching one or more teeth of the model data and the previous model data; applying, to the model data, geometric transformations of tooth positions to desired tooth positions; applying constraint optimizations to account for changes in tooth shapes between the previous model data and the model data; and modifying the model data based on the constraint optimizations.

The constraint optimizations may include collisions between neighboring teeth in an arch. The constraint optimizations may include inter-arch collisions. In some examples the constraint optimizations requires collisions not deeper than about 0.2 mm. The constraint optimizations may reduce shifts of teeth from position given by previous model data. The constraint optimizations may pull buccal cusps of lower posterior teeth into grooves of upper posterior teeth.

In any of these examples, the method may include updating a treatment plan for the patient using the modified model data and/or fabricating an orthodontic appliance (or a series of orthodontic appliances) based on the treatment plan.

Also described herein are methods comprising: detecting, in a processor, one or more dental attachments on a digital model of a patient's teeth, wherein the patient is undergoing a first treatment plan; determining, from a revised treatment plan, one or more of the one or more dental attachments to remove; and presenting, in a user interface, an image of the digital model of the patient's teeth with one or more markers visually indicating the one or more of the one or more dental attachments to remove.

Any of these methods may include adjusting the digital model of the patient's teeth to identify the one or more dental attachments. This may be done automatically as discussed above (e.g., in and/or by a processor), including any of these steps. For example, these methods may include receiving model data of the patient's teeth, the model data including one or more dental attachments and detecting, from the model data, the one or more attachments.

Presenting, in the user interface, the image of the digital model of the patient's teeth with one or more markers visually indicating the one or more of the one or more dental attachments to remove may include coloring the one or more dental attachments to remove an identifying color and/or encircling the one or more dental attachments.

Also described herein are non-transitory computer-readable medium comprising one or more computer-executable instructions that, when executed by at least one processor of a computing device, cause the computing device to perform any of the methods described herein.

Described herein are methods and apparatuses (e.g., systems, including software, hardware and/or firmware) for performing these methods. In general, these methods and apparatuses may improve the speed of treatment for removing attachments (e.g., obsolete or “white” attachments), and for generating treatment plans. For example, described herein are methods including: detecting, from a digital model of a patient's dentition, one or more attachments on the patient's dentition; copying at least a first portion of the digital model of the patient's dentition to create a first copy of the digital model of the patient's dentition; performing a user-input process on the digital model of the patient's dentition; performing, in parallel with the performance of the user-input process, the steps of: modifying the first copy of the digital model of the patient's dentition, and segmenting the modified first copy of the digital model; and segmenting the digital model of the patient's dentition based on the segmentation of the modified first copy of the digital model.

For example, described herein are method comprising: detecting, from a digital model of a patient's dentition, one or more attachments on the patient's dentition; copying a first portion of the digital model of the patient's dentition to create a first copy of the digital model of the patient's dentition; copying a second portion of the digital model of the patient's dentition to create a second copy of the digital model of the patient's dentition; performing a user-input process on the digital model of the patient's dentition, comprising reviewing and/or changing the detected one or more or attachments; performing, in parallel with the performance of the user-input process, the steps of: modifying either or both the first copy of the digital model of the patient's dentition and the second copy of the digital model of the patient's dentition to remove the detected one or more attachments, and segmenting the modified first copy of the digital model and the modified second copy of the digital model; repeating the steps of modifying the first copy and the second copy and segmenting the modified first copy and the modified second copy if the user-input process changes the detected one or more or attachments; and segmenting the digital model of a patient's dentition based on the segmentation of the modified first copy of the digital model and the modified second copy of the digital model.

As mentioned, also described herein is software for performing any of these methods. For example, described herein are non-transitory computer-readable medium comprising one or more computer-executable instructions that, when executed by at least one processor of a computing device, cause the computing device to perform the method of: detecting, from a digital model of a patient's dentition, one or more attachments on the patient's dentition; copying at least a first portion of the digital model of the patient's dentition to create a first copy of the digital model of the patient's dentition; performing a user-input process on the digital model of the patient's dentition; performing, in parallel with the performance of the user-input process, the steps of modifying the first copy of the digital model of the patient's dentition and segmenting the modified first copy of the digital model; and segmenting the digital model of the patient's dentition based on the segmentation of the modified first copy of the digital model.

Also described herein are systems for performing any of these methods. These systems may include one or more processors; a memory coupled to the one or more processors, the memory storing computer-executable instructions, that, when executed by the one or more processors, perform a computer-implemented method comprising: detecting, from a digital model of a patient's dentition, one or more attachments on the patient's dentition; copying at least a first portion of the digital model of the patient's dentition to create a first copy of the digital model of the patient's dentition; performing a user-input process on the digital model of the patient's dentition; performing, in parallel with the performance of the user-input process, the steps of modifying the first copy of the digital model of the patient's dentition and segmenting the modified first copy of the digital model; and segmenting the digital model of the patient's dentition based on the segmentation of the modified first copy of the digital model.

In any of these methods and apparatuses the first portion of the digital model of the patient's dentition may comprise an upper jaw of the patient or a portion thereof, and the (optional) second portion may comprise the lower jaw or portion thereof. For example, any of these methods and apparatuses may include copying a second portion of the digital model of the patient's dentition to create a second copy of the digital model of the patient's dentition, wherein modifying the first copy of the digital model of the patient's dentition also includes modifying the second copy of the digital model of the patient's dentition, further wherein segmenting the digital model of the patient's dentition is based on the segmentation of the modified first copy of the digital model and the modified second copy of the digital model.

Any of these methods and apparatuses may include performing the user-input process on the digital model of the patient's dentition including reviewing and/or changing the detected one or more or attachments. This step may include presenting a user interface and allowing the user to confirm or otherwise modify the attachment, or to modify the shape of the attachment and/or the underlying tooth.

The step of modifying the first copy of the digital model of the patient's dentition may comprise modifying the first copy to remove the detected one or more attachments.

Any of these methods and apparatuses may repeat the steps of modifying the first copy and segmenting the modified first copy if the user-input process changes the detected one or more or attachments.

In general, these methods and apparatuses may output the segmented digital model of a patient's dentition and/or may generate or modify a treatment plan from (e.g., using) the resulting segmented digital model of a patient's dentition.

Also described herein are methods and apparatuses (e.g., systems and devices, including software) for identifying a gingiva strip (in either or both the upper and lower jaws) in a 3D digital model of the patient's teeth (e.g., model data of a dental structure of a patient). These methods and apparatuses may also include asynchronous processing, which may improve the speed of treatment plan generation.

For example, described herein are methods including: performing one or more processing steps on a digital model of a patient's dentition; copying at least a portion of the digital model of the patient's dentition to create a copy the digital model of the patient's dentition; performing one or more user-input processes on the digital model of the patient's dentition; generating, in parallel with the performance of the one or more user-input processes, a gingiva strip from the copy of the digital model of the patient's dentition; comparing the digital model of the patient's dentition to the copy of the digital model of the patient's dentition; and modifying the digital model of the patient's dentition to include the gingiva strip if the copy of the digital model of the patient's dentition is substantially unchanged from the digital model of the patient's dentition, otherwise generating a second gingiva strip from the digital model of the patient's dentition and modifying the digital model of the patient's dentition to include the second gingiva strip.

For example, a method may include: performing a processing step on a digital model of the patient's dentition, wherein the processing step comprises modifying the digital model of the patient's dentition; copying a first portion of the digital model of the patient's dentition to create a copy of the patient's dentition; performing one or more user-input processes on the digital model of the patient's dentition, wherein the user-input processes comprises receiving one or more user inputs from a user interface; generating, in parallel with the performance of the one or more user-input processes, a gingiva strip from the copy of the digital model of the patient's dentition; comparing the digital model of the patient's dentition to the copy of the digital model of the patient's dentition by comparing a cyclic redundancy check (CRC) code for the digital model of the patient's dentition to a CRC code for the copy of the digital model of the patient's dentition; and modifying the digital model of the patient's dentition to include the gingiva strip if the copy of the digital model of the patient's dentition is substantially unchanged from the digital model of the patient's dentition, otherwise generating a second gingiva strip from the digital model of the patient's dentition and modifying the digital model of the patient's dentition to include the second gingiva strip.

Also described herein is software for performing these method, including non-transitory computer-readable media comprising one or more computer-executable instructions that, when executed by at least one processor of a computing device, cause the computing device to perform the method of: performing one or more processing steps on a digital model of a patient's dentition; copying at least a first portion of the digital model of the patient's dentition to create a copy of the digital model of the patient's dentition; performing one or more user-input processes on the digital model of the patient's dentition; generating, in parallel with the performance of the one or more user-input processes, a gingiva strip from the copy of the digital model of the patient's dentition; comparing the digital model of the patient's dentition to the copy of the digital model of the patient's dentition; and modifying the digital model of the patient's dentition to include the gingiva strip if the copy of the digital model of the patient's dentition is substantially unchanged from the digital model of the patient's dentition, otherwise generating a second gingiva strip from the digital model of the patient's dentition and modifying the digital model of the patient's dentition to include the second gingiva strip.

Also described herein are systems, which may include: one or more processors; a memory coupled to the one or more processors, the memory storing computer-program instructions, that, when executed by the one or more processors, perform a computer-implemented method comprising: performing one or more processing steps on a digital model of a patient's dentition; copying at least a first portion of the digital model of the patient's dentition to create a copy of the digital model of the patient's dentition; performing one or more user-input processes on the digital model of the patient's dentition; generating, in parallel with the performance of the one or more user-input processes, a gingiva strip from the copy of the digital model of the patient's dentition; comparing the digital model of the patient's dentition to the copy of the digital model of the patient's dentition; and modifying the digital model of the patient's dentition to include the gingiva strip if the copy of the digital model of the patient's dentition is substantially unchanged from the digital model of the patient's dentition, otherwise generating a second gingiva strip from the digital model of the patient's dentition and modifying the digital model of the patient's dentition to include the second gingiva strip.

In any of these methods, performing one or more processing steps on a digital model of the patient's dentition may comprise modifying the digital model of the patient's dentition. For example, modifying the digital model to add or remove features, modifying the digital model to move one or more teeth, and/or one or more of: determining collisions, identifying interproximal reductions, and/or modifying a clinical crown.

Any of these methods or apparatuses may include receiving the digital model of a patient's dentition.

In general, performing one or more user-input processes on the digital model of the patient's dentition may comprise receiving one or more user inputs from a user interface. For example, performing one or more user-input processes on the digital model of the patient's dentition may comprise one or more of: confirming a tooth axis, reviewing tooth position, modifying one or more settings, and reviewing automatically-generated comments.

Comparing the digital model of the patient's dentition to the copy of the digital model of the patient's dentition may comprise comparing a cyclic redundancy check (CRC) code for the digital model of the patient's dentition to a CRC code for the copy of the digital model of the patient's dentition. These methods and apparatuses may include calculating the CRC code for the copy of the digital model of the patient's dentition while generating the gingiva strip from the copy of the digital model of the patient's dentition.

As mentioned above, any of these methods and apparatuses may include generating a treatment plan from the modified digital model of the patient's dentition.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1A illustrates an exemplary tooth repositioning appliance or aligner that can be worn by a patient in order to achieve an incremental repositioning of individual teeth in the jaw, in accordance with some embodiments.

FIG. 1B illustrates a tooth repositioning system, in accordance with some embodiments.

FIG. 2 illustrates a 3D model data of a patient's dental structure including attachments, in accordance with some embodiments.

FIG. 3 illustrates a system for simulating and planning an orthodontic treatment, in accordance with some embodiments.

FIG. 4 illustrates a block diagram of an example system for automatic attachment detection and removal from 3D models, in accordance with some embodiments.

FIG. 5 illustrates a method of automatically detecting and removing attachments from 3D models, according to embodiments herein.

FIG. 6 illustrates a block diagram of an example system and workflow for automatic attachment detection and removal from 3D models, in accordance with some embodiments.

FIGS. 7A and 7B illustrate scan data of a patient's dentition with attachments, in accordance with some embodiments.

FIGS. 7C-D illustrate tooth model comparison, in accordance with some embodiments.

FIG. 8 illustrates an attachment plateau, in accordance with some embodiments.

FIGS. 9A-D illustrate diagrams showing attachment removal, in accordance with some embodiments.

FIG. 10A illustrates a control view which may provide an interface for confirming or rejecting detected attachments.

FIG. 10B illustrates attachments, as detected by systems and methods described herein.

FIG. 11 illustrates a method of building a secondary treatment plan for a same treatment goal based on a new intraoral scan, in accordance with some embodiments.

FIG. 12 shows a block diagram of an example computing system capable of implementing one or more embodiments described and/or illustrated herein, in accordance with some embodiments.

FIG. 13 shows a block diagram of an example computing network capable of implementing one or more of the embodiments described and/or illustrated herein, in accordance with some embodiments.

FIGS. 14A-14C illustrate examples of a 3D digital model of a patient's dentition, showing the dentition with white attachments (e.g., obsolete attachments) following identification (FIG. 14A), removal (FIG. 14B) and segmentation and identification of individual teeth (FIG. 14C).

FIG. 15 schematically illustrates an example of a method of asynchronous processing to remove attachments.

FIG. 16 schematically illustrates an example of a method of asynchronous processing to remove attachments.

FIG. 17A shows an example of a 3D digital model of a patient's dentition.

FIG. 17B illustrates an example of a gingiva strip portion of the 3D digital model of the patient's dentition shown in FIG. 17A.

FIG. 18 schematically illustrates an example of a method of asynchronously processing a 3D digital model of the patient's dentition to identify and process a gingiva strip within the 3D digital model.

FIG. 19 schematically illustrates an example of asynchronously processing a 3D digital model of the patient's dentition to process a gingiva strip within the 3D digital model.

### DETAILED DESCRIPTION

The following detailed description provides a better understanding of the features and advantages of the inventions described in the present disclosure in accordance with the embodiments disclosed herein. Although the detailed description includes many specific embodiments, these are provided by way of example only and should not be construed as limiting the scope of the inventions disclosed herein.

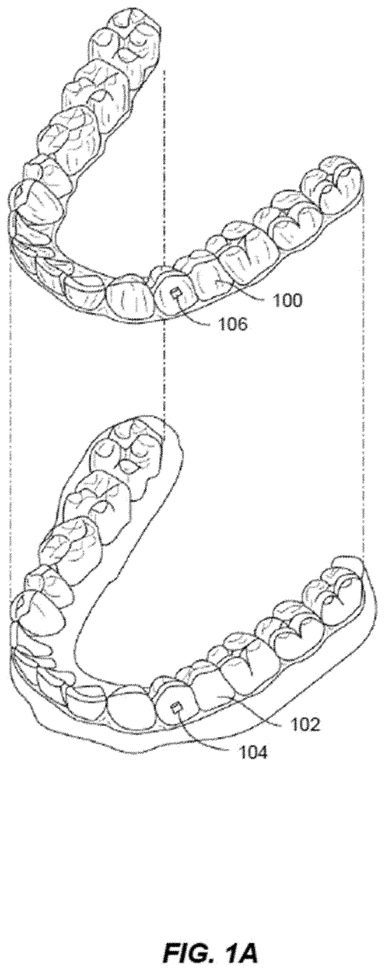

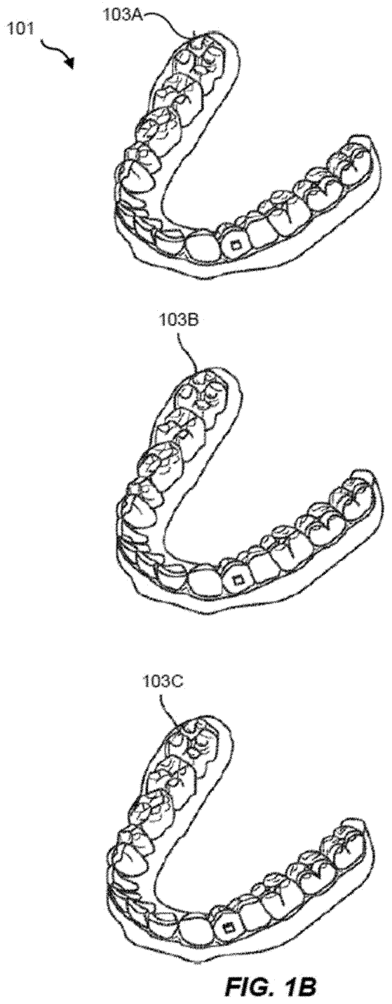

FIG. 1A illustrates an exemplary tooth repositioning appliance 100, such as an aligner that can be worn by a patient in order to achieve an incremental repositioning of individual teeth 102 in the jaw. The appliance can include a shell (e.g., a continuous polymeric shell or a segmented shell) having teeth-receiving cavities that receive and resiliently reposition the teeth. An appliance or portion(s) thereof may be indirectly fabricated using a physical model of teeth. For example, an appliance (e.g., polymeric appliance) can be formed using a physical model of teeth and a sheet of suitable layers of polymeric material. The physical model (e.g., physical mold) of teeth can be formed through a variety of techniques, including 3D printing. The appliance can be formed by thermoforming the appliance over the physical model. In some embodiments, a physical appliance is directly fabricated, e.g., using additive manufacturing techniques, from a digital model of an appliance. In some embodiments, the physical appliance may be created through a variety of direct formation techniques, such as 3D printing. An appliance can fit over all teeth present in an upper or lower jaw, or less than all of the teeth. The appliance can be designed specifically to accommodate the teeth of the patient (e.g., the topography of the tooth-receiving cavities matches the topography of the patient's teeth), and may be fabricated based on positive or negative models of the patient's teeth generated by impression, scanning, and the like. Alternatively, the appliance can be a generic appliance configured to receive the teeth, but not necessarily shaped to match the topography of the patient's teeth. In some cases, only certain teeth received by an appliance will be repositioned by the appliance while other teeth can provide a base or anchor region for holding the appliance in place as it applies force against the tooth or teeth targeted for repositioning. In some cases, some or most, and even all, of the teeth will be repositioned at some point during treatment. Teeth that are moved can also serve as a base or anchor for holding the appliance as it is worn by the patient. In some embodiments, no wires or other means will be provided for holding an appliance in place over the teeth. In some cases, however, it may be desirable or necessary to provide individual attachments or other anchoring elements 104 on teeth 102 with corresponding receptacles or apertures 106 in the appliance 100 so that the appliance can apply a selected force on the tooth. Exemplary appliances, including those utilized in the Invisalign® System, are described in numerous patents and patent applications assigned to Align Technology, Inc. including, for example, in U.S. Pat. Nos. 6,450,807, and 5,975,893, as well as on the company's website, which is accessible on the World Wide Web (see, e.g., the URL “invisalign.com”). Examples of tooth-mounted attachments suitable for use with orthodontic appliances are also described in patents and patent applications assigned to Align Technology, Inc., including, for example, U.S. Pat. Nos. 6,309,215 and 6,830,450.

FIG. 1B illustrates a tooth repositioning system 101 including a plurality of appliances 103A, 103B, 103C. Any of the appliances described herein can be designed and/or provided as part of a set of a plurality of appliances used in a tooth repositioning system. Each appliance may be configured so a tooth-receiving cavity has a geometry corresponding to an intermediate or final tooth arrangement intended for the appliance. The patient's teeth can be progressively repositioned from an initial tooth arrangement to a target tooth arrangement by placing a series of incremental position adjustment appliances over the patient's teeth. For example, the tooth repositioning system 101 can include a first appliance 103A corresponding to an initial tooth arrangement, one or more intermediate appliances 103B corresponding to one or more intermediate arrangements, and a final appliance 103C corresponding to a target arrangement. A target tooth arrangement can be a planned final tooth arrangement selected for the patient's teeth at the end of all planned orthodontic treatment. Alternatively, a target arrangement can be one of some intermediate arrangements for the patient's teeth during the course of orthodontic treatment, which may include various different treatment scenarios, including, but not limited to, instances where surgery is recommended, where interproximal reduction (IPR) is appropriate, where a progress check is scheduled, where anchor placement is best, where palatal expansion is desirable, where restorative dentistry is involved (e.g., inlays, onlays, crowns, bridges, implants, veneers, and the like), etc. As such, it is understood that a target tooth arrangement can be any planned resulting arrangement for the patient's teeth that follows one or more incremental repositioning stages. Likewise, an initial tooth arrangement can be any initial arrangement for the patient's teeth that is followed by one or more incremental repositioning stages.

Optionally, in cases involving more complex movements or treatment plans, it may be beneficial to utilize auxiliary components (e.g., features, accessories, structures, devices, components, and the like) in conjunction with an orthodontic appliance. Examples of such accessories include but are not limited to elastics, wires, springs, bars, arch expanders, palatal expanders, twin blocks, occlusal blocks, bite ramps, mandibular advancement splints, bite plates, pontics, hooks, brackets, headgear tubes, springs, bumper tubes, palatal bars, frameworks, pin- and-tube apparatuses, buccal shields, buccinator bows, wire shields, lingual flanges and pads, lip pads or bumpers, protrusions, divots, and the like. In some embodiments, the appliances, systems and methods described herein include improved orthodontic appliances with integrally formed features that are shaped to couple to such auxiliary components, or that replace such auxiliary components.

In some cases, after a patient has gone through treatment (e.g., primary order), the patient may require a second treatment (e.g., secondary order). When the doctor creates the secondary order, the doctor may request to remove some of the attachments placed in the primary order from the corresponding three-dimensional (3D) model of the patient's dental structure. The doctor may need to determine which attachments should be physically removed and which ones should be left on the patient's teeth before starting the second treatment so that the appliances for the secondary order properly fit. However, the 3D model (e.g., as used with the primary order) may need to be updated for the secondary order.

A typical workflow for the secondary order may begin with the doctor scanning the patient's dentition. The doctor may remove unneeded attachments from the patient's teeth (e.g., from the primary order) or the doctor may scan the patient's teeth with all of the attachments from the primary order. The doctor may then create a secondary order for additional appliances and fill a prescription. The prescription may include a request for a technician to remove some attachments from the primary order from the 3D model, although the prescription may omit such a request.

The technician may remove attachments from the scan if requested. To remove the attachments, the technician may manually alter the 3D model by adjusting surface contours of the corresponding teeth. The technician may then proceed with creating new final positions for teeth using data imported from the previous (e.g., primary) order. The technician may run staging and place new attachments for the secondary treatment in the new 3D model.

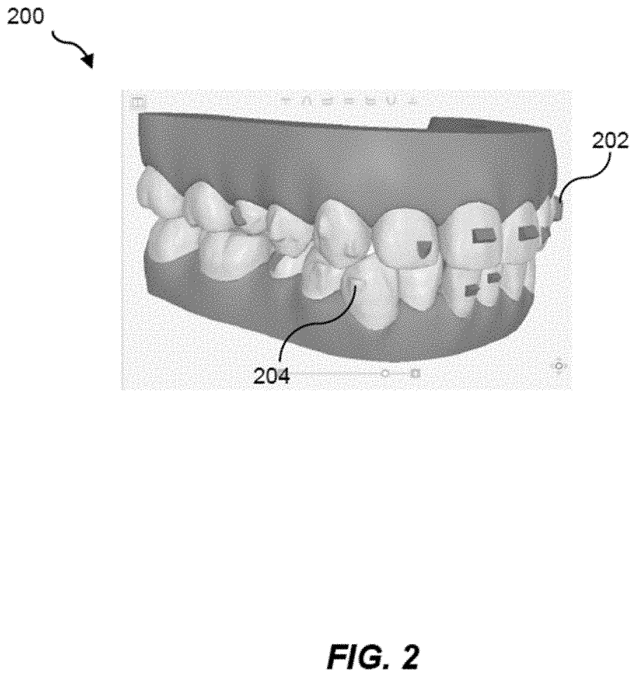

The doctor may view the new 3D model, an example of which is shown in FIG. 2. As seen in FIG. 2, model 200 of the patient's dental structure may include various attachments, such as attachment 202 and attachment 204. Attachment 202 may be a new attachment for the secondary order and displayed with a visual highlight, such as red shading. Attachment 204 may be an existing attachment to be reused for the secondary order (e.g., “white attachment”) and displayed as part of the corresponding tooth shape. However, model 200 may not show removed attachments. The doctor may need to refer to another interface, such as a PDF document, to see which attachments should be removed.

The present disclosure provides systems and methods for automatic attachment material detection and removal from 3D model data. The systems and methods provided herein may improve the functioning of a computing device by efficiently producing accurate 3D model data without requiring significantly more data or complex calculations. In addition, the systems and methods provided herein may improve the field of medical care by improving a digital workflow procedure by reducing costs of human time spent on processing, increasing efficiency via automation, and reducing potential errors. Moreover, the systems and methods provided herein may improve the field of 3D modeling of anatomy by improving detection and removal of structural features.

A “dental consumer,” as used herein, may include a person seeking assessment, diagnosis, and/or treatment for a dental condition (general dental condition, orthodontic condition, endodontic condition, condition requiring restorative dentistry, etc.). A dental consumer may, but need not, have agreed to and/or started treatment for a dental condition. A “dental patient” (used interchangeably with patient herein) as used herein, may include a person who has agreed to diagnosis and/or treatment for a dental condition. A dental consumer and/or a dental patient, may, for instance, be interested in and/or have started orthodontic treatment, such as treatment using one or more (e.g., a sequence of) aligners (e.g., polymeric appliances having a plurality of tooth-receiving cavities shaped to successively reposition a person's teeth from an initial arrangement toward a target arrangement).

A “dental professional” (used interchangeably with dentist, orthodontist, and doctor herein) as used herein, may include any person with specialized training in the field of dentistry, and may include, without limitation, general practice dentists, orthodontists, dental technicians, dental hygienists, etc. A dental professional may include a person who can assess, diagnose, and/or treat a dental condition. “Assessment” of a dental condition, as used herein, may include an estimation of the existence of a dental condition. An assessment of a dental condition need not be a clinical diagnosis of the dental condition. In some embodiments, an “assessment” of a dental condition may include an “image based assessment,” that is an assessment of a dental condition based in part or on whole on photos and/or images (e.g., images that are not used to stitch a mesh or form the basis of a clinical scan) taken of the dental condition. A “diagnosis” of a dental condition, as used herein, may include a clinical identification of the nature of an illness or other problem by examination of the symptoms. “Treatment” of a dental condition, as used herein, may include prescription and/or administration of care to address the dental conditions. Examples of treatments to dental conditions include prescription and/or administration of brackets/wires, clear aligners, and/or other appliances to address orthodontic conditions, prescription and/or administration of restorative elements to address bring dentition to functional and/or aesthetic requirements, etc.

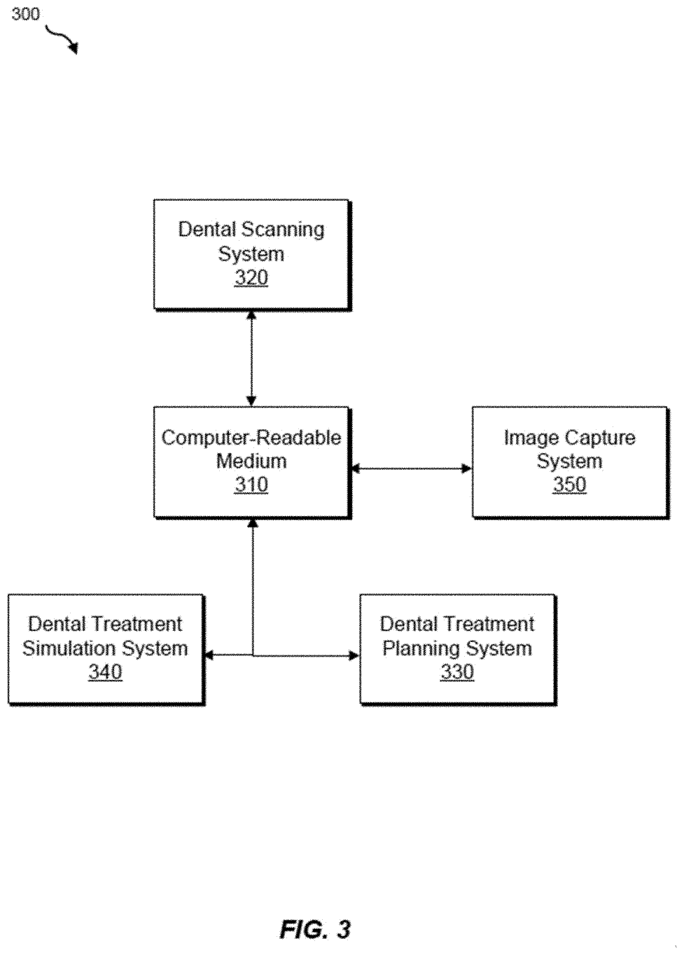

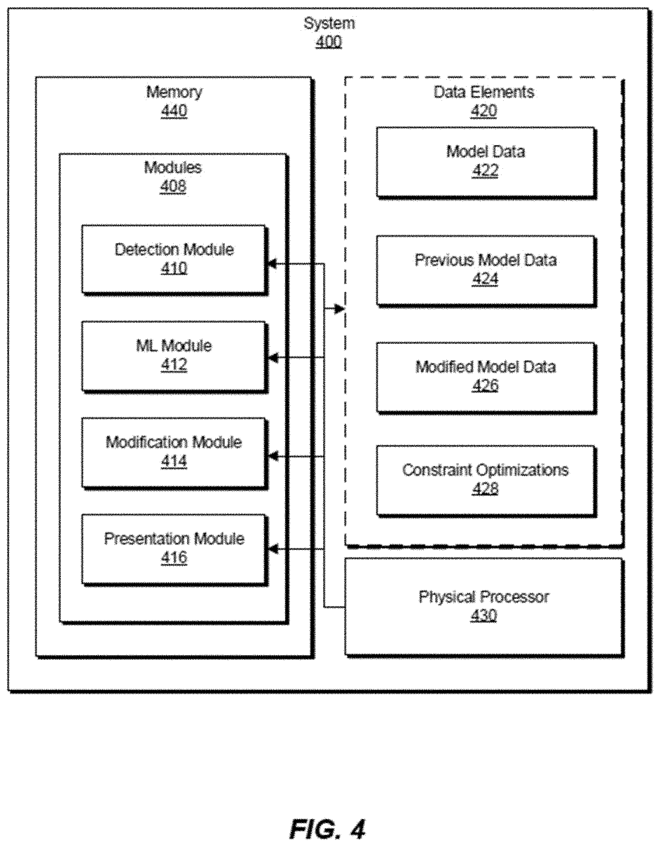

FIG. 3 shows a system 300 for simulating and planning an orthodontic treatment, in accordance with some embodiments. In the example of FIG. 3, the system 300 includes a computer-readable medium 310, a dental scanning system 320, a dental treatment planning system 330, a dental treatment simulation system 340, and an image capture system 350. One or more of the elements of the system 300 may include elements of such as those described with reference to the computer system shown in FIG. 4 and vice versa. One or more elements of system 300 may also include one or more computer readable media including instructions that when executed by a processor, for example, a processor of any of systems 320, 330, 340, and 350 cause the respective system or systems to perform the processes described herein.

Dental scanning system 320 may include a computer system configured to capture one or more scans of a patient's dentition. Dental scanning system 320 may include a scan engine for capturing 2D or 3D images of a patient. Such images may include images of the patient's teeth, face, and jaw, for example. The images may also include x-rays, computed tomography, magnetic resonance imaging (MRI), cone beam computed tomography (CBCT), cephalogram images, panoramic x-ray images, digital imaging and communication in medicine (DICOM) images, or other subsurface images of the patient. The scan engine may also capture 3D data representing the patient's teeth, face, gingiva, or other aspects of the patient.

Dental scanning system 320 may also include a 2D imaging system, such as a still or video camera, an x-ray machine, or other 2D imager. In some embodiments, dental scanning system 320 may also include a 3D imager, such as an intraoral scanner, an impression scanner, a tomography system, a cone beam computed tomography (CBCT) system, or other system as described herein, for example. Dental scanning system 320 and associated engines and imagers can be used to capture the model data for use in detecting attachments, as described herein. Dental scanning system 320 and associated engines and imagers can be used to capture the 2D and 3D images of a patient's face and dentition for use in building a 3D parametric model of the patient's teeth as described herein. Examples of parametric models of the patient's teeth suitable for incorporation in accordance with the present disclosure are describe in U.S. application Ser. No. 16/400,980, filed on May 1, 2019, entitled “Providing a simulated outcome of dental treatment on a patient”, published as US20200000551on Jan. 2, 2020, the entire disclosure of which is incorporated herein by reference.

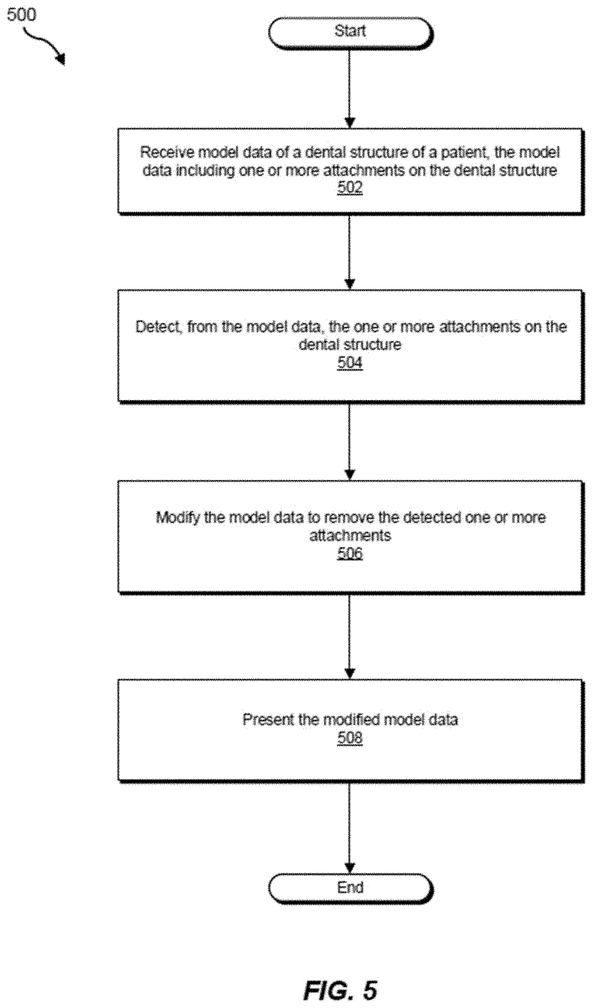

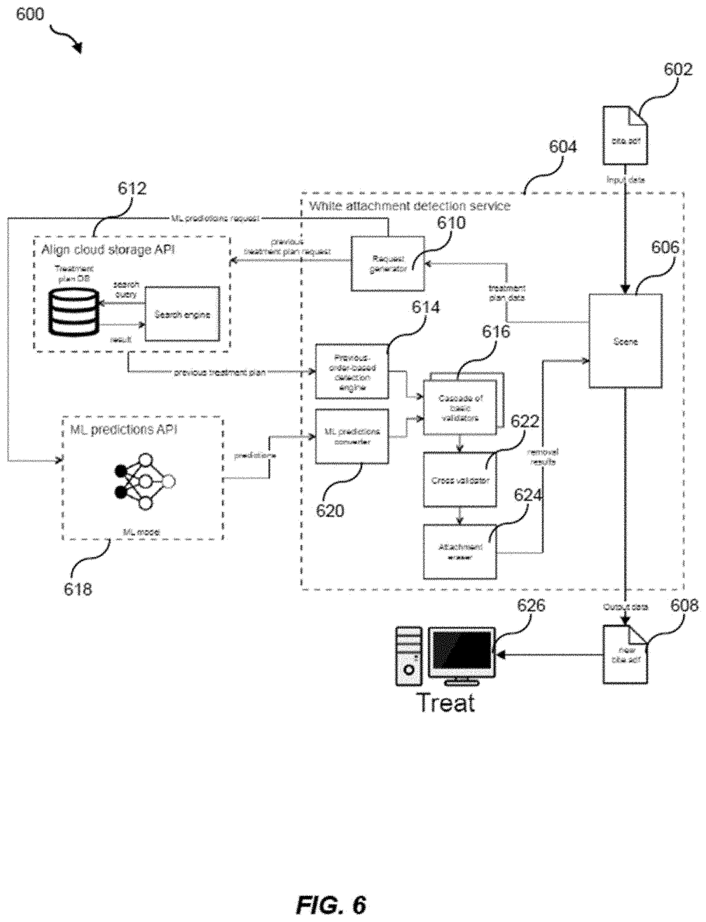

Dental treatment simulation system 340 may include a computer system configured to simulate one or more estimated and/or intended outcomes of a dental treatment plan. In some implementations, dental treatment simulation system 340 obtains photos and/or other 2D images of a consumer/patient. Dental treatment simulation system 340 may further be configured to determine tooth, lip, gingiva, and/or other edges related to teeth in the 2D image. As noted herein, dental treatment simulation system 340 may be configured to match tooth and/or arch parameters to tooth, lip, gingiva, and/or other edges. Dental treatment simulation system 340 may also render a 3D tooth model of the patient's teeth. Dental treatment simulation system 340 may gather information related to historical and/or idealized arches representing an estimated outcome of treatment. Dental treatment simulation system 340 may, in various implementations, insert, align, etc. the 3D tooth model with the 2D image of the patient in order to render a 2D simulation of an estimated outcome of orthodontic treatment. Dental treatment simulation system 340 may include a photo parameterization engine which may further include an edge analysis engine, an EM analysis engine, a course tooth alignment engine, and a 3D parameterization conversion engine. The dental treatment simulation system 340 may also include a parametric treatment prediction engine which may further include a treatment parameterization engine, a scanned tooth normalization engine, and a treatment plan remodeling engine. Dental treatment simulation system 340 and its associated engines may carry out the processes described herein, for example with reference to FIGS. 5, 6, and/or 11.

Dental treatment planning system 330 may include a computer system configured to implement treatment plans. Dental treatment planning system 330 may include a rendering engine and interface for visualizing or otherwise displaying the simulated outcome of the dental treatment plan. For example, the rendering engine may render the visualizations of the 3D models described herein. Dental treatment planning system 330 may also determine an orthodontic treatment plan for moving a patient's teeth from an initial position, for example, based in part on the 2D image of the patient's teeth, to a final position. Dental treatment planning system 330 may be operative to provide for image viewing and manipulation such that rendered images may be scrollable, pivotable, zoomable, and interactive. Dental treatment planning system 330 may include graphics rendering hardware, one or more displays, and one or more input devices. Some or all of dental treatment planning system 330 may be implemented on a personal computing device such as a desktop computing device or a handheld device, such as a mobile phone. In some embodiments, at least a portion of dental treatment planning system 330 may be implemented on a scanning system, such as dental scanning system 320. Image capture system 350 may include a device configured to obtain an image, including an image of a patient. The image capture system may comprise any type of mobile device (iOS devices, iPhones, iPads, iPods, etc., Android devices, portable devices, tablets), PCs, cameras (DSLR cameras, film cameras, video cameras, still cameras, etc.). In some implementations, image capture system 350 comprises a set of stored images, such as images stored on a storage device, a network location, a social media website, etc.

FIG. 4 is a block diagram of an example system 400 for automatic attachment detection and removal, in accordance with some embodiments. System 400 generally represents any type or form of computing device capable of reading computer-executable instructions. System 400 may be, for example, a desktop computer, a tablet computing device, a laptop, a smartphone, an augmented reality device, or other consumer device. Additional examples of system 400 include, without limitation, laptops, tablets, desktops, servers, cellular phones, Personal Digital Assistants (PDAs), multimedia players, embedded systems, wearable devices (e.g., smart watches, smart glasses, etc.), smart vehicles, smart packaging (e.g., active or intelligent packaging), gaming consoles, Internet-of-Things devices (e.g., smart appliances, etc.), variations or combinations of one or more of the same, and/or any other suitable computing device.

In addition, system 400 generally represents any type or form of computing device that is capable of storing and analyzing data. System 400 may include a backend database server for storing patient data and treatment data. Additional examples of system 400 include, without limitation, security servers, application servers, web servers, storage servers, and/or database servers configured to run certain software applications and/or provide various security, web, storage, and/or database services. Although illustrated as a single entity in FIG. 4, system 400 may include and/or represent a plurality of servers that work and/or operate in conjunction with one another.

As illustrated in FIG. 4, system 400 may include one or more memory devices, such as memory 440. Memory 440 generally represents any type or form of volatile or non-volatile storage device or medium capable of storing data and/or computer-readable instructions. In one example, memory 440 may store, load, execute in conjunction with physical processor(s) 430, and/or maintain one or more of modules 408. Examples of memory 440 include, without limitation, Random Access Memory (RAM), Read Only Memory (ROM), flash memory, Hard Disk Drives (HDDs), Solid-State Drives (SSDs), optical disk drives, caches, variations or combinations of one or more of the same, and/or any other suitable storage memory.

As illustrated in FIG. 4, system 400 may also include one or more physical processors, such as physical processor(s) 430. Physical processor(s) 430 generally represents any type or form of hardware-implemented processing unit capable of interpreting and/or executing computer-readable instructions. In one example, physical processor(s) 430 may access and/or modify one or more of modules 408 stored in memory 440. Additionally or alternatively, physical processor 430 may execute one or more of modules 408 to facilitate preamble phrase. Examples of physical processor(s) 430 include, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, and/or any other suitable physical processor.

Example system 400 in FIG. 4 may be implemented in a variety of ways. For example, all or a portion of example system 400 may represent portions of the systems in FIGS. 3, 6, 12, and/or 13.

As illustrated in FIG. 4, example system 200 may include one or more modules 408 for performing one or more tasks. As will be explained in greater detail below, modules 408 may include a detection module 410, a machine learning (“ML”) module 412, a modification module 414, and a presentation module 416. Although illustrated as separate elements, one or more of modules 408 in FIG. 4 may represent portions of a single module or application.

In certain embodiments, one or more of modules 408 in FIG. 4 may represent one or more software applications or programs that, when executed by a computing device, may cause the computing device to perform one or more tasks. For example, and as will be described in greater detail below, one or more of modules 408 may represent modules stored and configured to run on one or more computing devices, such as the devices illustrated in FIGS. 3, 6, 12, and/or 13. One or more of modules 408 in FIG. 4 may also represent all or portions of one or more special-purpose computers configured to perform one or more tasks.

As further illustrated in FIG. 4, system 400 may include data elements 420, including model data 422, previous model data 424, modified model data 426, and constraint optimizations 428. One or more of data elements 420 may be stored locally and/or retrieved from a remote storage, such as a datastore or database.

FIG. 5 is a flow diagram of an exemplary computer-implemented method 500 for automatically detecting and removing attachments from 3D model data. The steps shown in FIG. 5 may be performed by any suitable computer-executable code and/or computing system, including the system(s) illustrated in FIGS. 3, 6, 12, and/or 13. In one example, each of the steps shown in FIG. 5 may represent an algorithm whose structure includes and/or is represented by multiple sub-steps, examples of which will be provided in greater detail below.

As illustrated in FIG. 5, at step 502 one or more of the systems described herein may receive model data of a dental structure of a patient. The model data may include one or more attachments on the dental structure. For example, detection module 410 may receive model data 422 from, for example, a datastore, a remote server, from a scanning device, retrieved from local storage, etc. Model data 422 may include scan data of a patient's dental structure, particularly after a treatment. Model data 422 may include attachments from the previous treatment.

In some embodiments, the term “model data” may refer to three-dimensional data that may be scanned from a patient. Model data may be scanned by a scanning device, such as dental scanning system 320. Model data may correspond to the 3D data, 3D models, 3D tooth models, and/or patient dentition described herein.

FIG. 6 illustrates a workflow 600 for automatically detecting and removing attachment material from 3D model data. As illustrated in FIG. 6, input data 602, which may correspond to model data 422, may be provided to a detection service 604. In some implementations, computational overhead may be offloaded by having a separate service, such as detection service 604, for attachment detection and removal. Input data 602 may be received by a module 606, which may correspond to one or more of modules 408.

Turning back to FIG. 5, at step 504 one or more of the systems described herein may detect, from the model data, the one or more attachments on the dental structure. For example, detection module 410 may detect attachments on the dental structure in model data 422.

The systems described herein may perform step 504 in a variety of ways. In one example, detection module 410 may detect attachments based on a previous order. For example, in FIG. 6, module 606 may send treatment plan data to request generator 610. Request generator 610 may request previous treatment plan data from a database 612. Previous-order-based detection engine 614, which may correspond to detection module 410, may retrieve previous treatment plan data (e.g., previous model data 424) from database 612. The previous treatment plan data may include previous 3D model data of the patient's dental structure and may include attachments.

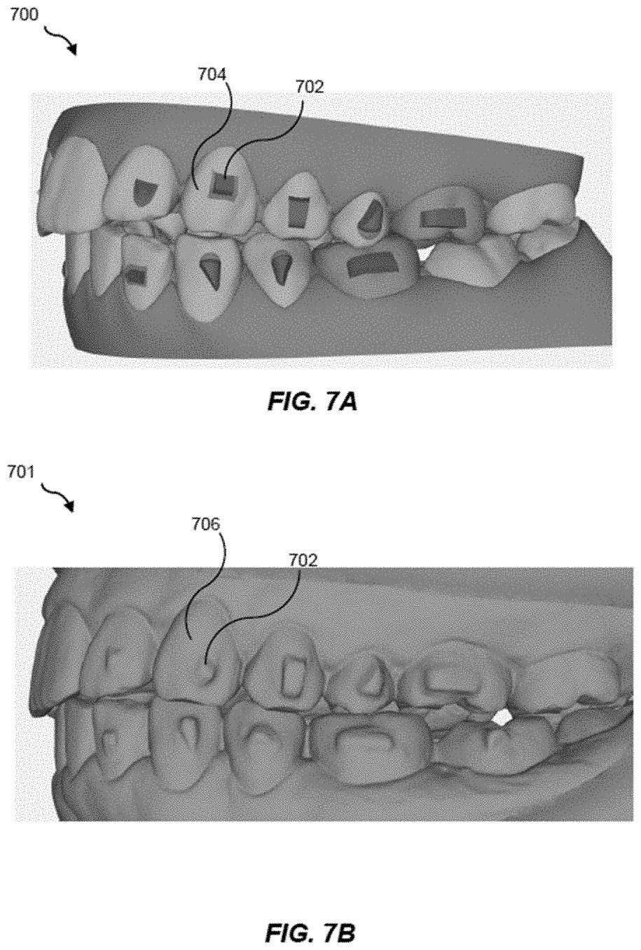

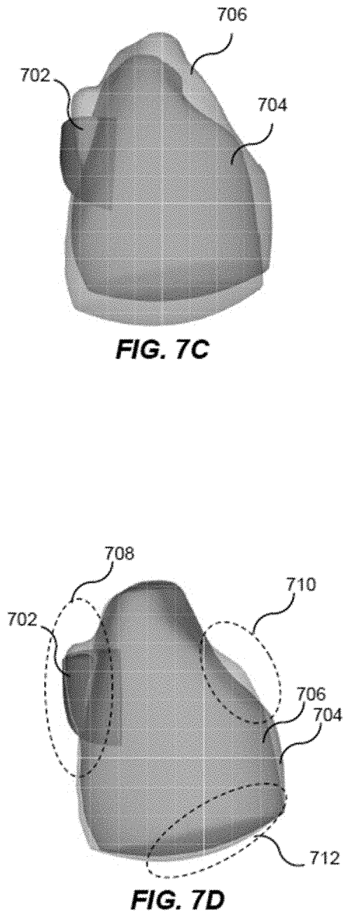

Detection engine 614 may match one or more teeth of the previous model data with respective one or more teeth of the model data for comparison. Detection engine 614 may identify one or more previous attachments from the previous model data. As illustrated in FIG. 7A, a model 700 of a patient's dental structure may include an attachment 702 on a tooth model 704. Model 700, which may correspond to previous model data 424, may include attachment 702 as part of the previous order. However, when scanning the patient's dental structure having attachment 702, attachment 702 may be recognized as part of the anatomy and rendered as a monolithic part of tooth models, as seen in FIG. 7B. FIG. 7B illustrates a model 701, which may correspond to model data 422, having attachment 702 integrated as a part of tooth model 706 as an extrusion.

As illustrated in FIG. 7C, after matching tooth model 704 with tooth model 706, detection engine 614 may find a matching transform between. In other words, overlaying each tooth model with the corresponding tooth model from the previous treatment may minimize shape deviations to improve attachment detection. As illustrated in FIG. 7D, once overlaid, shape discrepancies 712 and 710 may not be related to attachment placement. Based on the placement of attachment 702 from previous model data, detection engine 614 may detect shape discrepancy 708 corresponding to attachment 702. Thus, detection engine 614 may identify attachment 702 based on the shape discrepancy between tooth model 704 and tooth model 706. Detection engine 614 may provide its detected attachments to validators 616.

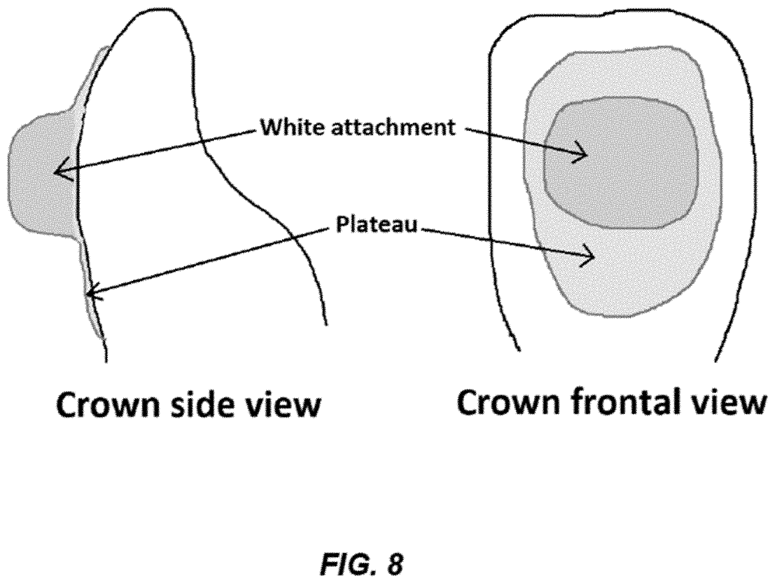

Previous-order-based detection may have certain limitations. For example, trimmed teeth or teeth with significant anatomy discrepancies, such as interproximal reduction, may cause incorrect teeth matching may provide poor detection. In addition, excessive attachment material around the attachment template (e.g., an “attachment plateau” surrounding the actual attachment, see, e.g., FIG. 8) may not be properly detected.

In some examples, detection module 410 may detect attachments using a machine learning model, such as machine learning module 412, which may be a machine learning model trained to detect attachments on tooth models. For example, in FIG. 6, request generator 610 may make a ML predictions request to an ML model 618, which may correspond to machine learning module 412. ML model 618 may provide predictions of extra material on tooth models to an ML predictions converter 620. ML predictions converter 620 may identify the extra material as an attachment and provide its detected attachments to validators 616.

Although using an ML model may advantageously not require additional data (e.g., previous model data 424) for making predictions, and may be trained, based on a training dataset, to better detect attachment plateau, using the ML model may have certain limitations. For instance, predicted areas may have unclear borders such as small leaks. The ML model may confuse regular distortions with white attachments due to similar forms. Additionally, distance predictions (e.g., between attachment surfaces and “true” tooth surfaces) may be over- or under-estimated, which may produce crater shapes when removing attachments, as further described herein.

Validators 616 may include various prediction validations based on, for example, statistical distribution of certain attachment characteristics such as area or maximum depth. Additionally and/or alternatively, detection service 604 may apply cross-validation via cross validator 622 to accept or reject predicted attachments based on results from multiple approaches. For example, cross validator 622 may receive a first set of potential attachments from previous-order-based-detection engine 614, and a second set of potential attachments from ML model 618. Cross validation may reduce false-positive detections.

Cross validator 622 may use one or more rules. For example, cross validator 622 may discard potential attachments of the first or second set of potential attachments that are close to interproximal or occlusal tooth areas if another of the first or second set of potential attachments lacks matching potential attachments. Cross validator 622 may discard, from the ML set of potential attachments, potential attachments for areas that do not have significant deviations in the model data compared to the previous model data. Cross validator 622 may discard, from the previous-order-based set of potential attachments, potential attachments having a small distance to a corresponding tooth surface that do not intersect with potential attachments of the second set of potential attachments.

Detection service 604 may employ various other techniques for improving results from on multiple approaches. Detection service 604 may apply detection supplementation using area or distance predictions from one approach to improve results of another approach. For example, the previous-order-based approach may improve “attachment plateau” detection by expanding detected areas intersecting with attachment areas predicted by the ML model, as illustrated in FIG. 8. The ML model may use reference shapes to correct distance predictions.

Returning to FIG. 5, at step 506 one or more of the systems described herein may modify the model data to remove the detected one or more attachments. For example, modification module 414 may modify model data 422 by removing the detected attachments to produce modified model data 426.

The systems described herein may perform step 506 in a variety of ways. In one example, for each of the detected one or more attachments modification module 414 may calculate a depth from the attachment to a corresponding tooth based on the previous model data and adjust a surface of the attachment towards a direction inside the tooth based on the calculated depth. Modification module 414 moving scan vertices inside a detected area corresponding to the attachment in the direction inside the tooth. Alternatively and/or additionally, modification module 414 may use predicted depths from ML module 412. ML module 412 may predict depths to a predicted “true” tooth surface.

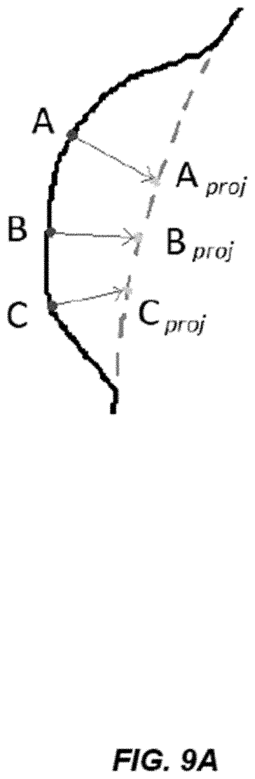

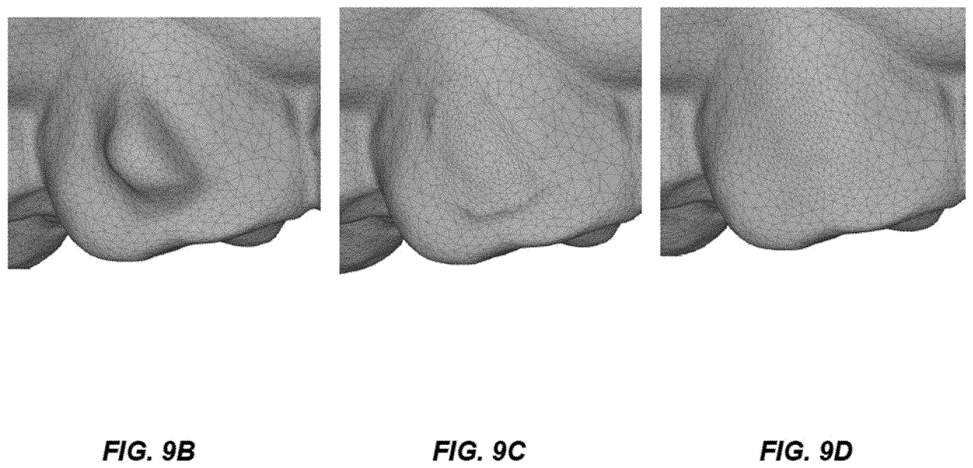

FIG. 9A illustrates how vertices A, B, and C may be moved to Aproj, Bproj, and Cproj, respectively. FIG. 9B illustrates scan vertices of a detected attachment and FIG. 9C illustrates the scan vertices moved to remove the attachment. In some examples, modification module 414 may perform additional refinement to further smooth out the tooth surface, as illustrated in FIG. 9D.

Turning to FIG. 6, attachment eraser 624, which may correspond to modification module 414, may remove detected attachments based on the cross-validated attachments provided by cross validator 622. The removal results from attachment eraser 624 may be received and processed by module 606 to produce output data 608, which may correspond to modified model data 426.

Returning to FIG. 5, at step 508 one or more of the systems described herein may present the modified model data. For example, presentation module 416 may present or otherwise provide for presentation modified model data 426.

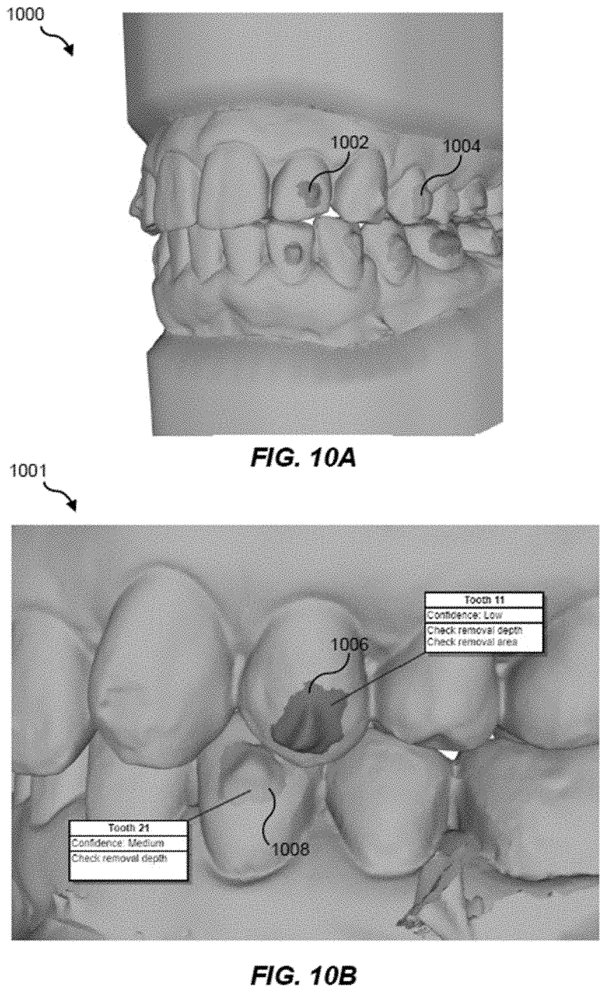

The systems described herein may perform step 508 in a variety of ways. In one example, a preview view may display teeth with attachments removed, as in FIG. 9D. In another example, presentation module 416 may display visual indicators of the removed one or more attachments. FIG. 10A illustrates a control view 1000 which may provide an interface for confirming or rejecting detected attachments. For example, an attachment 1002, which may be displayed with a visual indicator for rejection such as red color, may correspond to a detected attachment that should be kept. An attachment 1004, which may be displayed with a visual indicator for confirmation such as green color, may correspond to a detected attachment that should be removed.

Referring to FIG. 6, output data 608 may be provided to an interface 626 (e.g., a computing device as described herein) to allow the doctor to confirm and/or reject detected attachments and make other adjustments as needed.

In some examples, presentation module 416 may display, with corresponding confidence values, a plurality of attachment removal options based on the detected one or more attachments. FIG. 10B illustrates an attachment 1006 and an attachment 1008, as detected by systems and methods described herein. Each attachment may be displayed with a visual indicator of confidence value, such as colors indicating low, medium, and high confidence. In some examples, as in FIG. 10B, additional information may be presented, for instance to provide further explanation to confidence values as well as provide recommendations to facilitate confirming or rejecting a detected attachment. In some examples, multiple variations of the same attachment may be presented, which may vary in dimension, shape, etc.

The confidence values may be based on one or more metrics, such as a degree of similarity between corresponding attachments detected via multiple detection approaches. For example, attachments detected by multiple approaches (e.g., previous-order-based approach and ML model-based approach as describe herein) and further having a high similarity in area and distance predictions may be associated with a high confidence. Attachments detected by multiple approaches but having significant differences in a dimension (e.g., one of area predictions, distance predictions, etc.) may be associated with a medium confidence. Attachments detected by multiple approaches but having significant differences in more than one dimension (e.g., one or more of area predictions, distance predictions, etc.) may be associated with a low confidence.

In some examples, method 500 may further include updating a treatment plan for the patient using the modified model data. For example, the doctor may update and finalize the secondary order.

In some examples, method 500 may further include fabricating an orthodontic appliance based on the treatment plan. For example, once the secondary order is confirmed, an appliance may be fabricated using the treatment plan.

Although method 500 is presented as a sequence of steps, in some examples, the steps of method 500 may be repeated as needed to automatically detect and remove attachments from the 3D model data. In addition, although method 500 is described herein with respect to a secondary order, in other examples, the steps of method 500 may be applied to other cases of updating a treatment.

### Secondary Treatment Plan

In some orthodontics cases, a doctor may send a patient's dentition scans in the middle of a treatment and request a treatment to be built that starts from the scan and results in the same final position as the previous treatment. However, if the teeth from the new scan are put into their position from the previous scan, the resulting position may have clinically inacceptable collisions between teeth due to, for instance, scanning error. Such collisions may require manual corrections by CAD designers or other technicians before presenting to the doctor.

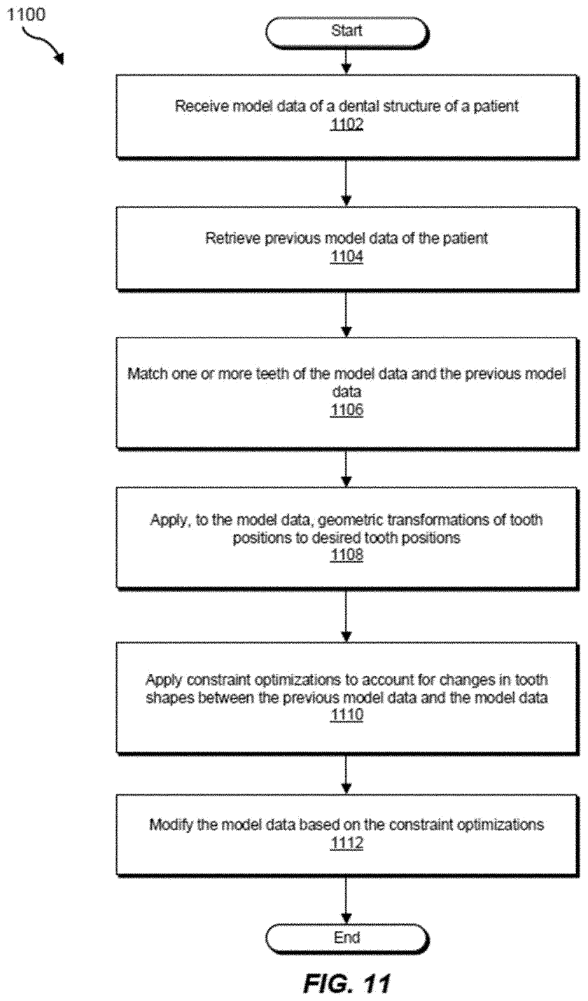

FIG. 11 is a flow diagram of an exemplary computer-implemented method 1100 for building a secondary treatment plan for the same treatment goal based on the new intraoral scan. The steps shown in FIG. 11 may be performed by any suitable computer-executable code and/or computing system, including the system(s) illustrated in FIGS. 3, 6, 12, and/or 13. In one example, each of the steps shown in FIG. 11 may represent an algorithm whose structure includes and/or is represented by multiple sub-steps, examples of which will be provided in greater detail below.

As illustrated in FIG. 11, at step 1102 one or more of the systems described herein may receive a model data of a dental structure of a patient. For example, detection module 410 may receive model data 422 from local and/or remote storage, a scanning device, etc.

At step 1104 one or more of the systems described herein may retrieve a previous model data of the patient. For example, detection module 410 may retrieve previous model data 424 from local and/or remote storage.

At step 1106 one or more of the systems described herein may match one or more teeth of the model data and the previous model data. For example, detection module 410 may match teeth of model data 422 with teeth of previous model data 424, similar to FIG. 7C.

At step 1108 one or more of the systems described herein may apply, to the model data, geometric transformations of tooth positions to desired tooth positions. For example, detection module 410 may apply geometric transformations to model data 422 and/or previous model data 424, similar to FIG. 7D.

At step 1110 one or more of the systems described herein may apply constraint optimizations to account for changes in tooth shapes between the previous model data and the model data. For example, modification module 414 may apply constraint optimizations 428.

The systems described herein may perform step 1110 in a variety of ways. In one example, a first group of constraint optimizations may relate to collisions between neighboring teeth in an arch. By default, normal contacts between neighboring teeth may be require. However, if there was a space between teeth in the primary final position, modification module 414 may keep the space. If there was an IPR between these teeth in the primary final position, modification module 414 may check if the position after previous model data 424 is closer to IPR or to a contact and creates an IPR or contact accordingly.

In some examples, a second group of constraints may relate to inter-arch collisions. The constraint optimization may require collisions not deeper than 0.2 mm except for pairs of teeth with deeper collisions (e.g., between about 0.2 and about 0.7 mm) in the final positions of the primary case. In such cases, the collisions may not be deeper than the final position of the primary case.

In addition, the constraint optimizations may be applied to two groups of targets. A first group of targets may minimize or otherwise reduce shits of teeth from the position provided by previous model data 424. A second group of targets may relate to occlusion contacts. The second group of targets may involve pulling buccal cusps of lower posterior teeth into grooves of upper posterior teeth.

As illustrated in FIG. 11, at step 1112 one or more of the systems described herein may modify the model data based on the constraint optimizations. For example, modification module 414 may modify model data 422 to produce modified model data 426.

In some examples, method 1100 may further include updating a treatment plan for the patient using the modified model data. In some examples, method 1100 may further include fabricating an orthodontic appliance based on the treatment plan. Although method 1100 is presented as a sequence of steps, in some examples, the steps of method 1100 may be repeated as needed.

### Computing System

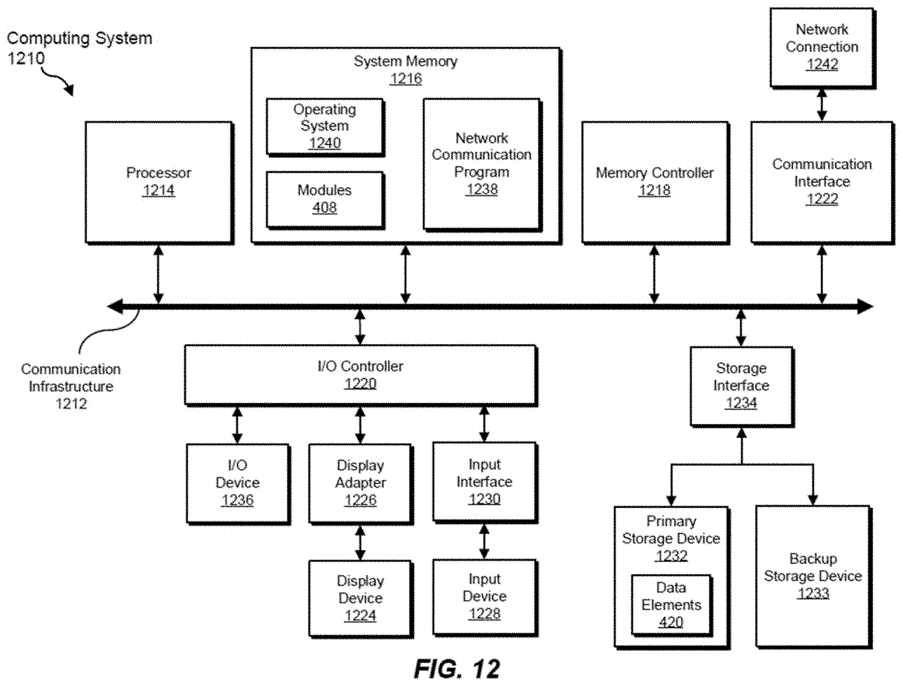

FIG. 12 is a block diagram of an example computing system 1210 capable of implementing one or more of the embodiments described and/or illustrated herein. For example, all or a portion of computing system 1210 may perform and/or be a means for performing, either alone or in combination with other elements, one or more of the steps described herein (such as one or more of the steps illustrated in FIGS. 5, 6, 11, 15, 16, 18 and/or 19). All or a portion of computing system 1210 may also perform and/or be a means for performing any other steps, methods, or processes described and/or illustrated herein.

Computing system 1210 broadly represents any single or multi-processor computing device or system capable of executing computer-readable instructions. Examples of computing system 1210 include, without limitation, workstations, laptops, client-side terminals, servers, distributed computing systems, handheld devices, or any other computing system or device. In its most basic configuration, computing system 1210 may include at least one processor 1214 and a system memory 1216.

Processor 1214 generally represents any type or form of physical processing unit (e.g., a hardware-implemented central processing unit) capable of processing data or interpreting and executing instructions. In certain embodiments, processor 1214 may receive instructions from a software application or module. These instructions may cause processor 1214 to perform the functions of one or more of the example embodiments described and/or illustrated herein.

System memory 1216 generally represents any type or form of volatile or non-volatile storage device or medium capable of storing data and/or other computer-readable instructions. Examples of system memory 1216 include, without limitation, Random Access Memory (RAM), Read Only Memory (ROM), flash memory, or any other suitable memory device. Although not required, in certain embodiments computing system 1210 may include both a volatile memory unit (such as, for example, system memory 1216) and a non-volatile storage device (such as, for example, primary storage device 1232, as described in detail below). In one example, one or more of modules 408 from FIG. 4 may be loaded into system memory 1216.

In some examples, system memory 1216 may store and/or load an operating system 1240 for execution by processor 1214. In one example, operating system 1240 may include and/or represent software that manages computer hardware and software resources and/or provides common services to computer programs and/or applications on computing system 1210. Examples of operating system 1240 include, without limitation, LINUX, JUNOS, MICROSOFT WINDOWS, WINDOWS MOBILE, MAC OS, APPLE'S IOS, UNIX, GOOGLE CHROME OS, GOOGLE'S ANDROID, SOLARIS, variations of one or more of the same, and/or any other suitable operating system.

In certain embodiments, example computing system 1210 may also include one or more components or elements in addition to processor 1214 and system memory 1216. For example, as illustrated in FIG. 12, computing system 1210 may include a memory controller 1218, an Input/Output (I/O) controller 1220, and a communication interface 1222, each of which may be interconnected via a communication infrastructure 1212. Communication infrastructure 1212 generally represents any type or form of infrastructure capable of facilitating communication between one or more components of a computing device. Examples of communication infrastructure 1212 include, without limitation, a communication bus (such as an Industry Standard Architecture (ISA), Peripheral Component Interconnect (PCI), PCI Express (PCIe), or similar bus) and a network.

Memory controller 1218 generally represents any type or form of device capable of handling memory or data or controlling communication between one or more components of computing system 1210. For example, in certain embodiments memory controller 1218 may control communication between processor 1214, system memory 1216, and I/O controller 1220 via communication infrastructure 1212.

I/O controller 1220 generally represents any type or form of module capable of coordinating and/or controlling the input and output functions of a computing device. For example, in certain embodiments I/O controller 1220 may control or facilitate transfer of data between one or more elements of computing system 1210, such as processor 1214, system memory 1216, communication interface 1222, display adapter 1226, input interface 1230, and storage interface 1234.

As illustrated in FIG. 12, computing system 1210 may also include at least one display device 1224 coupled to I/O controller 1220 via a display adapter 1226. Display device 1224 generally represents any type or form of device capable of visually displaying information forwarded by display adapter 1226. Similarly, display adapter 1226 generally represents any type or form of device configured to forward graphics, text, and other data from communication infrastructure 1212 (or from a frame buffer, as known in the art) for display on display device 1224.

As illustrated in FIG. 12, example computing system 1210 may also include at least one input device 1228 coupled to I/O controller 1220 via an input interface 1230. Input device 1228 generally represents any type or form of input device capable of providing input, either computer or human generated, to example computing system 1210. Examples of input device 1228 include, without limitation, a keyboard, a pointing device, a speech recognition device, variations or combinations of one or more of the same, and/or any other input device.

Additionally or alternatively, example computing system 1210 may include additional I/O devices. For example, example computing system 1210 may include I/O device 1236. In this example, I/O device 1236 may include and/or represent a user interface that facilitates human interaction with computing system 1210. Examples of I/O device 1236 include, without limitation, a computer mouse, a keyboard, a monitor, a printer, a modem, a camera, a scanner, a microphone, a touchscreen device, variations or combinations of one or more of the same, and/or any other I/O device.

Communication interface 1222 broadly represents any type or form of communication device or adapter capable of facilitating communication between example computing system 1210 and one or more additional devices. For example, in certain embodiments communication interface 1222 may facilitate communication between computing system 1210 and a private or public network including additional computing systems. Examples of communication interface 1222 include, without limitation, a wired network interface (such as a network interface card), a wireless network interface (such as a wireless network interface card), a modem, and any other suitable interface. In at least one embodiment, communication interface 1222 may provide a direct connection to a remote server via a direct link to a network, such as the Internet. Communication interface 1222 may also indirectly provide such a connection through, for example, a local area network (such as an Ethernet network), a personal area network, a telephone or cable network, a cellular telephone connection, a satellite data connection, or any other suitable connection.

In certain embodiments, communication interface 1222 may also represent a host adapter configured to facilitate communication between computing system 1210 and one or more additional network or storage devices via an external bus or communications channel. Examples of host adapters include, without limitation, Small Computer System Interface (SCSI) host adapters, Universal Serial Bus (USB) host adapters, Institute of Electrical and Electronics Engineers (IEEE) 1394 host adapters, Advanced Technology Attachment (ATA), Parallel ATA (PATA), Serial ATA (SATA), and External SATA (eSATA) host adapters, Fibre Channel interface adapters, Ethernet adapters, or the like. Communication interface 1222 may also allow computing system 1210 to engage in distributed or remote computing. For example, communication interface 1222 may receive instructions from a remote device or send instructions to a remote device for execution.

In some examples, system memory 1216 may store and/or load a network communication program 1238 for execution by processor 1214. In one example, network communication program 1238 may include and/or represent software that enables computing system 1210 to establish a network connection 1242 with another computing system (not illustrated in FIG. 12) and/or communicate with the other computing system by way of communication interface 1222. In this example, network communication program 1238 may direct the flow of outgoing traffic that is sent to the other computing system via network connection 1242. Additionally or alternatively, network communication program 1238 may direct the processing of incoming traffic that is received from the other computing system via network connection 1242 in connection with processor 1214.

Although not illustrated in this way in FIG. 12, network communication program 1238 may alternatively be stored and/or loaded in communication interface 1222. For example, network communication program 1238 may include and/or represent at least a portion of software and/or firmware that is executed by a processor and/or Application Specific Integrated Circuit (ASIC) incorporated in communication interface 1222.

As illustrated in FIG. 12, example computing system 1210 may also include a primary storage device 1232 and a backup storage device 1233 coupled to communication infrastructure 1212 via a storage interface 1234. Storage devices 1232 and 1233 generally represent any type or form of storage device or medium capable of storing data and/or other computer-readable instructions. For example, storage devices 1232 and 1233 may be a magnetic disk drive (e.g., a so-called hard drive), a solid state drive, a floppy disk drive, a magnetic tape drive, an optical disk drive, a flash drive, or the like. Storage interface 1234 generally represents any type or form of interface or device for transferring data between storage devices 1232 and 1233 and other components of computing system 1210.

In certain embodiments, storage devices 1232 and 1233 may be configured to read from and/or write to a removable storage unit configured to store computer software, data, or other computer-readable information. Examples of suitable removable storage units include, without limitation, a floppy disk, a magnetic tape, an optical disk, a flash memory device, or the like. Storage devices 1232 and 1233 may also include other similar structures or devices for allowing computer software, data, or other computer-readable instructions to be loaded into computing system 1210. For example, storage devices 1232 and 1233 may be configured to read and write software, data, or other computer-readable information. Storage devices 1232 and 1233 may also be a part of computing system 1210 or may be a separate device accessed through other interface systems.

Many other devices or subsystems may be connected to computing system 1210. Conversely, all of the components and devices illustrated in FIG. 12 need not be present to practice the embodiments described and/or illustrated herein. The devices and subsystems referenced above may also be interconnected in different ways from that shown in FIG. 12. Computing system 1210 may also employ any number of software, firmware, and/or hardware configurations. For example, one or more of the example embodiments disclosed herein may be encoded as a computer program (also referred to as computer software, software applications, computer-readable instructions, or computer control logic) on a computer-readable medium. The term “computer-readable medium,” as used herein, generally refers to any form of device, carrier, or medium capable of storing or carrying computer-readable instructions. Examples of computer-readable media include, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

The computer-readable medium containing the computer program may be loaded into computing system 1210. All or a portion of the computer program stored on the computer-readable medium may then be stored in system memory 1216 and/or various portions of storage devices 1232 and 1233. When executed by processor 1214, a computer program loaded into computing system 1210 may cause processor 1214 to perform and/or be a means for performing the functions of one or more of the example embodiments described and/or illustrated herein. Additionally or alternatively, one or more of the example embodiments described and/or illustrated herein may be implemented in firmware and/or hardware. For example, computing system 1210 may be configured as an Application Specific Integrated Circuit (ASIC) adapted to implement one or more of the example embodiments disclosed herein.

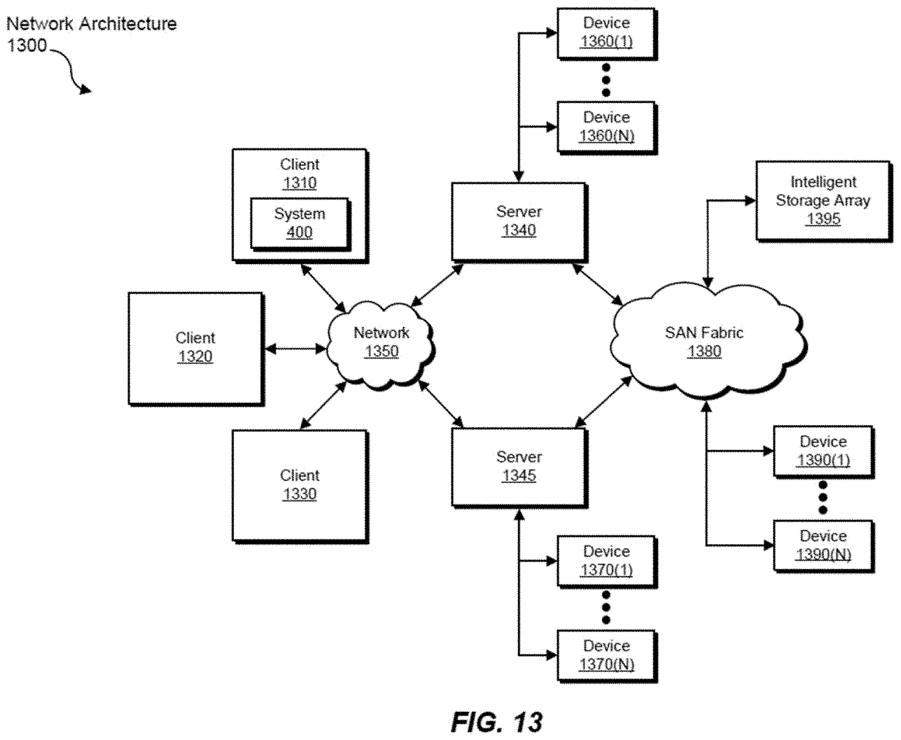

FIG. 13 is a block diagram of an example network architecture 1300 in which client systems 1310, 1320, and 1330 and servers 1340 and 1345 may be coupled to a network 1350. As detailed above, all or a portion of network architecture 1300 may perform and/or be a means for performing, either alone or in combination with other elements, one or more of the steps disclosed herein (such as one or more of the steps illustrated in FIG. 5, 6, and/or 11). All or a portion of network architecture 1300 may also be used to perform and/or be a means for performing other steps and features set forth in the instant disclosure.

Client systems 1310, 1320, and 1330 generally represent any type or form of computing device or system, such as example computing system 1210 in FIG. 12. Similarly, servers 1340 and 1345 generally represent computing devices or systems, such as application servers or database servers, configured to provide various database services and/or run certain software applications. Network 1350 generally represents any telecommunication or computer network including, for example, an intranet, a WAN, a LAN, a PAN, or the Internet. In one example, client systems 1310, 1320, and/or 1330 and/or servers 1340 and/or 1345 may include all or a portion of the systems in FIGS. 3, 6, 12, and/or 13.

As illustrated in FIG. 13, one or more storage devices 1360(1)-(N) may be directly attached to server 1340. Similarly, one or more storage devices 1370(1)-(N) may be directly attached to server 1345. Storage devices 1360(1)-(N) and storage devices 1370(1)-(N) generally represent any type or form of storage device or medium capable of storing data and/or other computer-readable instructions. In certain embodiments, storage devices 1360(1)-(N) and storage devices 1370(1)-(N) may represent Network-Attached Storage (NAS) devices configured to communicate with servers 1340 and 1345 using various protocols, such as Network File System (NFS), Server Message Block (SMB), or Common Internet File System (CIFS).

Servers 1340 and 1345 may also be connected to a Storage Area Network (SAN) fabric 1380. SAN fabric 1380 generally represents any type or form of computer network or architecture capable of facilitating communication between a plurality of storage devices. SAN fabric 1380 may facilitate communication between servers 1340 and 1345 and a plurality of storage devices 1390(1)-(N) and/or an intelligent storage array 1395. SAN fabric 1380 may also facilitate, via network 1350 and servers 1340 and 1345, communication between client systems 1310, 1320, and 1330 and storage devices 1390(1)-(N) and/or intelligent storage array 1395 in such a manner that devices 1390(1)-(N) and array 1395 appear as locally attached devices to client systems 1310, 1320, and 1330. As with storage devices 1360(1)-(N) and storage devices 1370(1)-(N), storage devices 1390(1)-(N) and intelligent storage array 1395 generally represent any type or form of storage device or medium capable of storing data and/or other computer-readable instructions.

In certain embodiments, and with reference to example computing system 1210 of FIG. 12, a communication interface, such as communication interface 1222 in FIG. 12, may be used to provide connectivity between each client system 1310, 1320, and 1330 and network 1350. Client systems 1310, 1320, and 1330 may be able to access information on server 1340 or 1345 using, for example, a web browser or other client software. Such software may allow client systems 1310, 1320, and 1330 to access data hosted by server 1340, server 1345, storage devices 1360(1)-(N), storage devices 1370(1)-(N), storage devices 1390(1)-(N), or intelligent storage array 1395. Although FIG. 13 depicts the use of a network (such as the Internet) for exchanging data, the embodiments described and/or illustrated herein are not limited to the Internet or any particular network-based environment.

In at least one embodiment, all or a portion of one or more of the example embodiments disclosed herein may be encoded as a computer program and loaded onto and executed by server 1340, server 1345, storage devices 1360(1)-(N), storage devices 1370(1)-(N), storage devices 1390(1)-(N), intelligent storage array 1395, or any combination thereof. All or a portion of one or more of the example embodiments disclosed herein may also be encoded as a computer program, stored in server 1340, run by server 1345, and distributed to client systems 1310, 1320, and 1330 over network 1350.

As detailed above, computing system 1210 and/or one or more components of network architecture 1300 may perform and/or be a means for performing, either alone or in combination with other elements, one or more steps of an example method for automatically detecting and removing attachments.

While the foregoing disclosure sets forth various embodiments using specific block diagrams, flowcharts, and examples, each block diagram component, flowchart step, operation, and/or component described and/or illustrated herein may be implemented, individually and/or collectively, using a wide range of hardware, software, or firmware (or any combination thereof) configurations. In addition, any disclosure of components contained within other components should be considered example in nature since many other architectures can be implemented to achieve the same functionality.

In some examples, all or a portion of example systems in FIGS. 3, 6, 12, and/or 13 may represent portions of a cloud-computing or network-based environment. Cloud-computing environments may provide various services and applications via the Internet. These cloud-based services (e.g., software as a service, platform as a service, infrastructure as a service, etc.) may be accessible through a web browser or other remote interface. Various functions described herein may be provided through a remote desktop environment or any other cloud-based computing environment.

In various embodiments, all or a portion of an example system in FIGS. 3, 6, 12, and/or 13 may facilitate multi-tenancy within a cloud-based computing environment. In other words, the software modules described herein may configure a computing system (e.g., a server) to facilitate multi-tenancy for one or more of the functions described herein. For example, one or more of the software modules described herein may program a server to enable two or more clients (e.g., customers) to share an application that is running on the server. A server programmed in this manner may share an application, operating system, processing system, and/or storage system among multiple customers (i.e., tenants). One or more of the modules described herein may also partition data and/or configuration information of a multi-tenant application for each customer such that one customer cannot access data and/or configuration information of another customer.

According to various embodiments, all or a portion of an example system in FIGS. 3, 6, 12, and/or 13 may be implemented within a virtual environment. For example, the modules and/or data described herein may reside and/or execute within a virtual machine. As used herein, the term “virtual machine” generally refers to any operating system environment that is abstracted from computing hardware by a virtual machine manager (e.g., a hypervisor). Additionally or alternatively, the modules and/or data described herein may reside and/or execute within a virtualization layer. As used herein, the term “virtualization layer” generally refers to any data layer and/or application layer that overlays and/or is abstracted from an operating system environment. A virtualization layer may be managed by a software virtualization solution (e.g., a file system filter) that presents the virtualization layer as though it were part of an underlying base operating system. For example, a software virtualization solution may redirect calls that are initially directed to locations within a base file system and/or registry to locations within a virtualization layer.

In some examples, all or a portion of example systems in FIGS. 3, 6, 12, and/or 13 may represent portions of a mobile computing environment. Mobile computing environments may be implemented by a wide range of mobile computing devices, including mobile phones, tablet computers, e-book readers, personal digital assistants, wearable computing devices (e.g., computing devices with a head-mounted display, smartwatches, etc.), and the like. In some examples, mobile computing environments may have one or more distinct features, including, for example, reliance on battery power, presenting only one foreground application at any given time, remote management features, touchscreen features, location and movement data (e.g., provided by Global Positioning Systems, gyroscopes, accelerometers, etc.), restricted platforms that restrict modifications to system-level configurations and/or that limit the ability of third-party software to inspect the behavior of other applications, controls to restrict the installation of applications (e.g., to only originate from approved application stores), etc. Various functions described herein may be provided for a mobile computing environment and/or may interact with a mobile computing environment.

In addition, all or a portion of example systems in FIGS. 3, 6, 12, and/or 13 may represent portions of, interact with, consume data produced by, and/or produce data consumed by one or more systems for information management. As used herein, the term “information management” may refer to the protection, organization, and/or storage of data. Examples of systems for information management may include, without limitation, storage systems, backup systems, archival systems, replication systems, high availability systems, data search systems, virtualization systems, and the like.

In some embodiments, all or a portion of example systems in FIGS. 3, 6, 12, and/or 13 may represent portions of, produce data protected by, and/or communicate with one or more systems for information security. As used herein, the term “information security” may refer to the control of access to protected data. Examples of systems for information security may include, without limitation, systems providing managed security services, data loss prevention systems, identity authentication systems, access control systems, encryption systems, policy compliance systems, intrusion detection and prevention systems, electronic discovery systems, and the like.

### Asynchronous Segmentation for Removal of Attachments

In some cases the methods and apparatuses described herein may include asynchronous (e.g., parallel) handling of one or more portions of the processes for modifying the model data of a dental structure of a patient refers equivalently to digital model of a patient's dentition. For example, the asynchronous processing may be used to segment (which may include in some cases re-segmenting or further segmenting) a 3D digital model of the patient's dentition. In particular, this segmentation may refer to segmentation of the teeth, including in particular the outer surface of the teeth where the attachments were previously positioned.

As described above, an attachment is an object on tooth that may have been created on previous treatment and may be removed during a secondary treatment. A user (e.g., doctor, technician, etc.) may ask to remove white attachments for additional treatment. Software, including a user interface, may be used as described above to automatically or semi-automatically identify and also to review the detected white attachments and accept removal for those one that detected correctly. When the user accepts the results, the attachments may be removed from the scan a segmentation process may begin. Segmentation may include recreation of tooth model shapes based on, e.g., a renewed scan surface. In current practice, segmentation may take a noticeable amount of time (e.g., about 15 seconds) and the user (e.g., technician, dental practitioner, etc.) may not be able to further process the case during that time, including not further modifying the model data of a dental structure of a patient (e.g., the digital model of the patient's dentition).

As described herein, the methods and apparatuses for processing a digital model of the patient's dentition, including but not limited to for processing the removal of attachments, may include asynchronously performing segmentation even before the digital model on which the segmentation is to be performed is completed. This approach may save a small, but significant amount of time on each case, e.g., approximately 10 seconds of scan segmentation time, after removal of the attachments. Over the course of the many hundreds and thousands of cases that may be processed, including remotely processed, this reduction in processing time may result in a substantial overall time and cost savings. The methods and apparatuses may increase the speed of a scan segmentation after the identification of attachments as described above, using asynchronously prepared data.

Once the method or apparatus has detected, from the digital model, one or more attachments on the patient's dentition, asynchronous processing may concurrently allow the user to both review and/or modify the attachment detection and to remove the attachments from one or more copies of the 3D model, and to segment the one or more copies of the 3D model. If the user further modifies the 3D model, including or in particular modifying the attachments to be removed or the manner or the remaining tooth surface once removed, the process of removing the attachment(s) and segmenting the copy or copies of the 3D model may be restarted. Once the user is done reviewing and/or modifying the attachments, e.g., in a user interface (a process referred to herein as a user-input process), the copy or copies of the 3D model may be used to segment the original 3D model of the patient's dentition. Thus, one or more customized copies of the 3D model (“scene”) may be created, and all asynchronous calculations may be performed with this copy/copies.

The methods and apparatuses described herein may begin asynchronous processing of the 3D model using the identified attachments before the results are finalized. By introducing “White attachments review” as part of a user interface, the calculation can be started (the beginning of the segmentation) early, and the resulting segmentation can be held until the results are required, e.g., at the end of the user-input process.

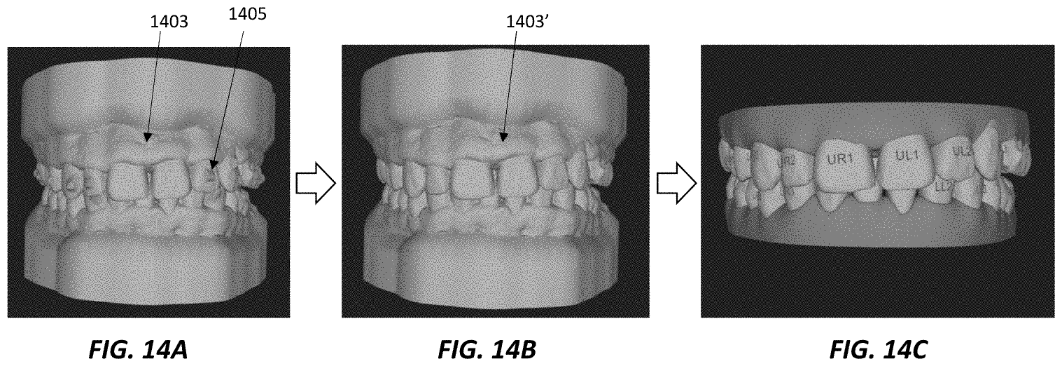

The use of asynchronous processing for white attachments segmentation may be particularly helpful because segmentation may be quite slow, and white attachments segmentation may have a limited number of scan changes (e.g., remove\restore white attachment). For example, FIG. 14A-14C illustrates the process of detecting attachments (white attachments), removing and segmenting. In FIG. 14A a 3D digital model of the patient's dentition 1403 is shown following an automatic process for identifying attachments 1405, as described above. Thereafter the attachments are removed (as shown in FIG. 14B) and the resulting revised 3D digital model 1403′ is segmented to identify individual teeth (and/or gingiva) as shown in FIG. 14C.

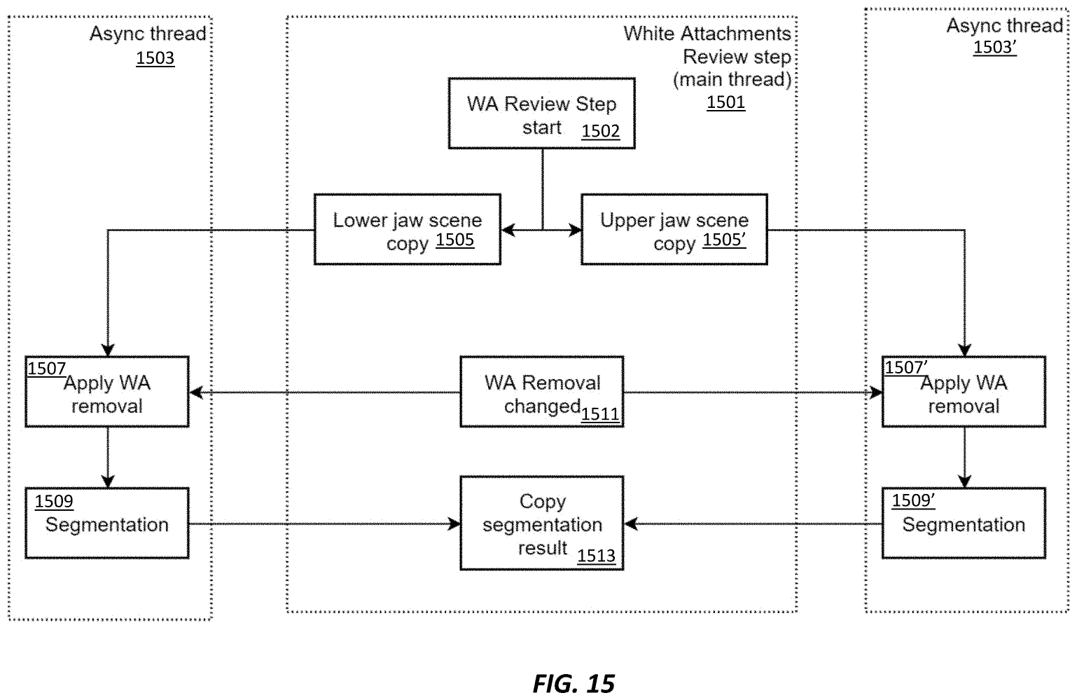

FIG. 15 shows an example of a method (which may be embodied as an apparatus, such as a system). In FIG. 15, following the automatic or semi-automatic identification of attachments in a 3D digital model, a review process 1501 (“review thread”) may begin. A copy of all or a portion of the 3D digital model of the patient's dentition (“scene”) may be made. In FIG. 15, a first copy of the lower jaw scene 1505 is made and a second copy, of the upper jaw scene 1505′ is also made. These copies are then used in two separate asynchronous threads 1503, 1503′ and processed concurrently with the use review of the attachments (“main thread” 1501). In the asynchronous threads, the processor (or a separate processor) may act on the copy of the 3D digital model, for example, removing the attachment(s) 1507, 1507′ and segmenting the dental model (e.g., into individual teeth) 1509, 1509′. Meanwhile, the user may review and/or make changes to the original 3D digital model (“scene”) including removing or changing the attachment(s) 1511. This may be done through a user interface. If changes are made, the modified 3D digital model may be transmitted (e.g., as a new copy or as modifications to the original copy) to the asynchronous thread(s) 1503, 1503′ and the new or updated 3D digital models may be re-processed, e.g., removing the attachments(s) 1507, 1507′ and segmenting 1509, 1509′. Once the user-input process in the main thread is completed, at least with respect to the attachments, the segmentation results from the asynchronous threads may be used to segment the main (e.g., original) 3D digital model 1513. For example, the segmentation of the upper jaw (in the first copy) may be copied into the upper jaw portion of the 3D digital model.

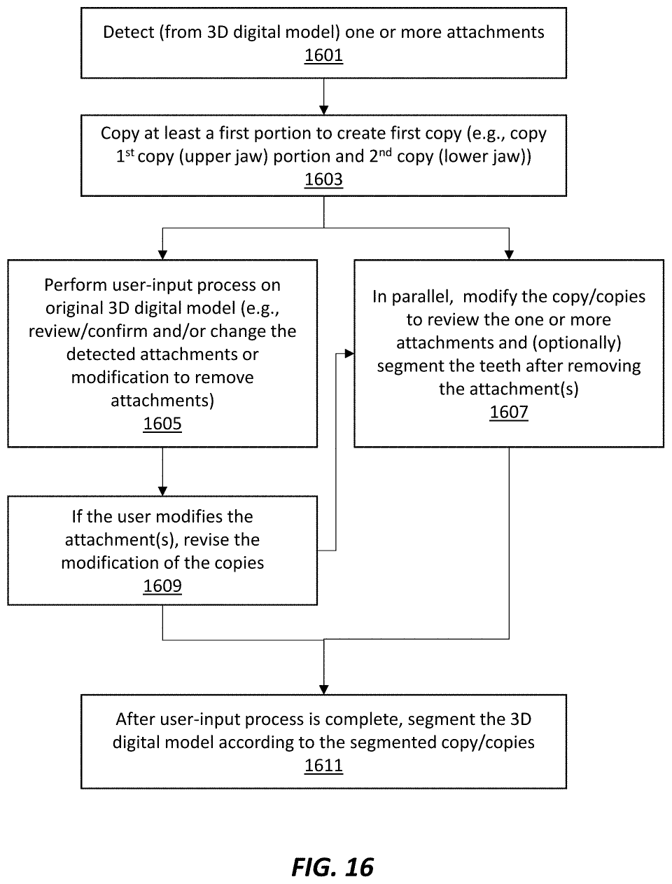

FIG. 16 illustrates another example of a method as described herein. In this example, the method (or an apparatus configured to perform the method) may detect, e.g., from a 3D digital model of the patient's dentition, one or more attachments 1601, as described above. The method may then include copying at least a first portion of the 3D digital model to create a first copy (e.g., of the upper or lower jaw). In some examples two copies are created; for example, each copy may contain only one jaw. This may allow the apparatus or to perform calculation in two asynchronous thread for each jaw independently. All detected attachments may be marked as applied for removal, e.g., during the asynchronous processing, and segmentation calculation may be performed. In FIG. 16, the user may concurrently perform one or more user-input process on the original 3D digital model (or in some examples, on another copy of the original 3D digital model), including using a user interface 1605. As shown in FIG. 16, in parallel the copy/copies of the 3D digital model may be modified as described above, using the identified attachments, and segmenting 1607.

If the user (e.g., technician, physician, dental professional, etc.) changes the attachments selection, asynchronous operation may restart with an updated scan (3D digital model) after the previous one was finished. This may happen independently for each jaw and/or region(s) of the jaw. Once the user-input process is complete the 3D digital model may be segmented using the copy/copies, for example, by copying the segmentation of these copies into the original 3D digital model 1611.

### Asynchronous Calculation of Gingiva Strip

The methods and apparatuses described herein may also or alternatively be used to identify and form a gingiva strip as part of the 3D digital model of the patient's dentition (e.g., the model data of a dental structure of a patient). The gingival line is a thin line around the patient's teeth which may be calculated as part of a process of the 3D digital model and may be based on jaw scan and used to create gingiva for the model.

The gingiva strip is a thin line around the teeth which may be used later to create gingiva. A gingiva strip may be created from a jaw scan for both jaws, e.g., as part of a 3D digital model of a patient's dentition. This processes, including treatment planning, using the digital model of the teeth may benefit from a smaller size of the digital model, by removing portions of the gingiva outside of the gingiva strip from the model.

The calculation of the gingiva strip may be performed during the processing of the 3D digital model prior to transferring (e.g., “porting”) the digital model to other modules for further treatment planning. Traditionally, a number of such processing steps may be performed during a combination of automatic and user-input steps. The calculation of the gingiva strip may take a relatively brief, but significant amount of time (e.g., around 5 seconds). This additional time may become significant when aggregated across multiple cases, and when a user is forced to delay further processing because of this calculation time, which may be irritating and disruptive. The methods and apparatuses described herein may apply asynchronously processing to determine a gingiva strip and apply the determined gingiva strip in a manner that may prevent the user from having to wait (e.g., for 5 seconds or more) during the process (e.g., during a “porting” process).

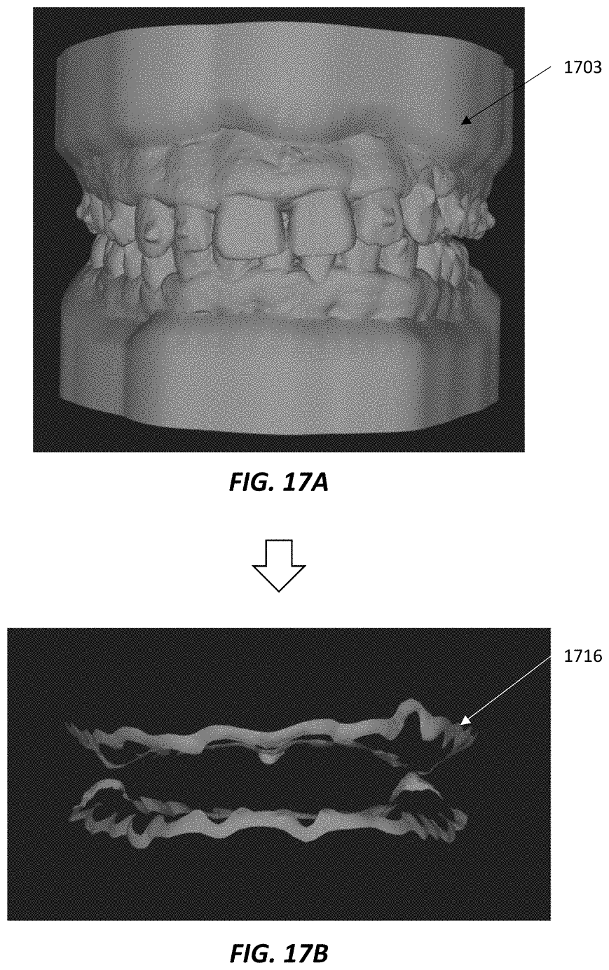

As mentioned above, The calculation of the gingiva strip may begin asynchronously before the results are needed by the process. FIG. 17A shows an example of a 3D digital model of a patient's dentition 1703, including teeth, gingiva and palatal regions. The 3D digital model may be processed to estimate a gingiva line 1716, as shown in FIG. 17B, in isolation from the rest of the patient's dentition. The gingiva line may be identified from the 3D digital model and may be segmented (e.g., grouped) as a specific portion or region of the 3D digital model.

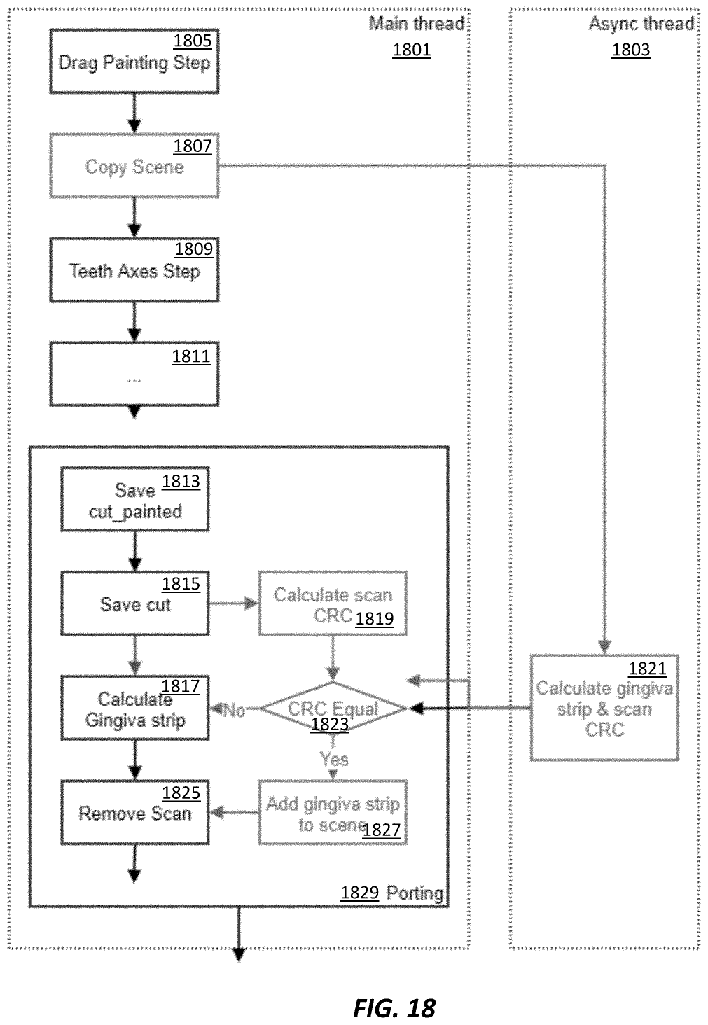

As mentioned, this may be done as part of a process using asynchronous processing. FIG. 18 illustrates an example. In FIG. 18 the primary or main thread 1801 may occur concurrently with one or more asynchronous threads 1803, including asynchronous threading to identify a gingiva line. Early in the main thread process the 3D digital model (“scene”) may be copied 1807; all or just a portion of the 3D digital model may be copied. One or more processing steps on the 3D digital model may be performed prior to copying the scene 1805. Following the copying step, additional steps, both user-input steps and autonomous steps may be performed in parallel with the calculation of the gingiva strip 1821. For example, as shown in FIG. 18 the main thread may include determining axes of the teeth 1809, and additional steps 1811, such as adding or modifying a clinical crown, etc. In FIG. 18, a sub-set of the main thread may be performed as part of, or in preparation for transferring the prepared 3D digital model to for developing a treatment plan (“porting” 1829). At a minimum this sub-set of the main thread may include (as one of the final steps) calculating a gingiva strip 1817. However, this step may instead by replaced with the gingiva strip calculated during the asynchronous thread, if the copy of the 3D digital model of the patient's dentition substantially matches the 3D digital model of the patient's dentition that was/is operated on during the main thread.

For example, in FIG. 18, the method may include as part of the main thread, a step of cutting 1813 and saving a modified version of the 3D digital model 1815. The 3D digital model, or this main-thread modified version of the 3D digital model may be scanned to determine a cyclic redundancy check (CRC) code 1819. The CRC code may be compared to a CRC code generated during the asynchronous thread (in parallel) from the copy of the 3D digital model used to generate the gingiva strip. The CRC does may be based on just the portion of the 3D model that is relevant to the gingiva (gingiva strip). If the 3D model of the main thread is substantially the same as the copy of the 3D model used to estimate the gingiva strip from the asynchronous thread (e.g., by comparing the CRC codes 1823) then the gingiva strip determined using the copy of the 3D digital model in the asynchronous process may be used as part of the primary 3D digital model 1827, otherwise the gingiva strip may be calculated from the primary 3D digital model of the main thread 1817. In some examples, the gingiva strip from the asynchronous process may replace the gingiva strip 1825.

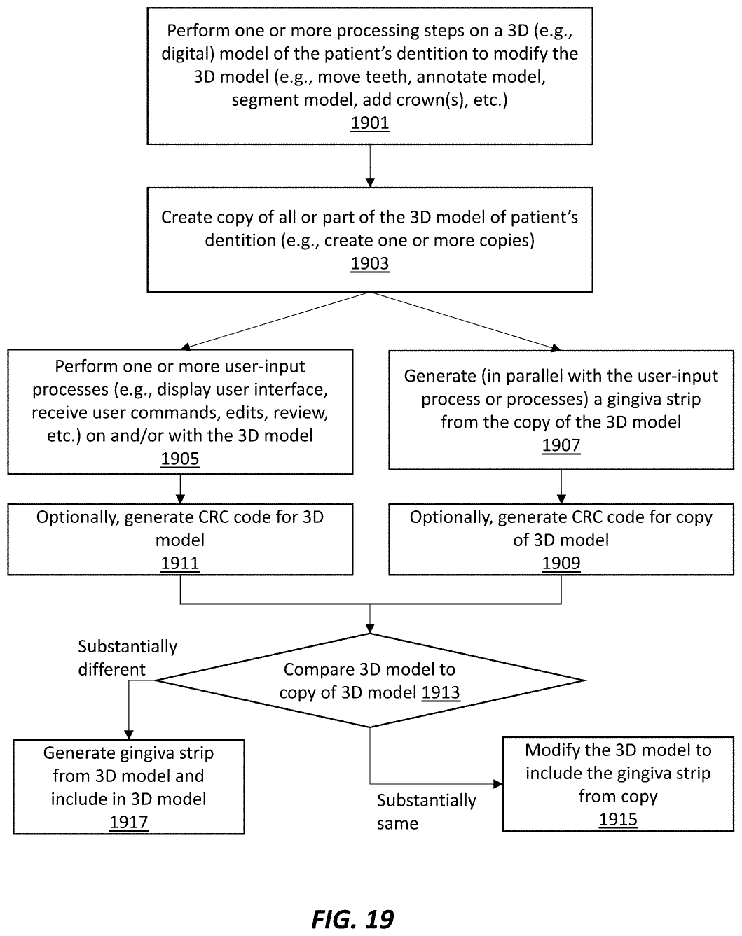

FIG. 19 illustrates another example of a method of determining a gingiva strip using an asynchronous process. In FIG. 19, the prior to initialing the asynchronous thread (e.g., determining the gingiva strip) one or more processes may be performed on the digital model of the patient's dentition 1901. Optionally, for example, the model of the patient's dentition may be modified by moving the teeth, annotating the model, segmenting all or a portion of the model, adding and/or modifying clinical crowns, etc.

A copy of all or portion (e.g., the relevant portion) of the 3D model of the patient's dentition may be made 1903 for use in the asynchronous thread. This may be referred to as a scene copy. For example, the scene (e.g., the 3D digital model) may have only required objects for current algorithm, all other objects may not be copied, which may save copy time. Required objects may be predetermined or may be selected manually. Scene components (e.g., portions of the 3D digital model) may be distinguished and/or isolated from other objects such as: main scene objects, global objects, GUI objects. In some examples, digital models may include connections between objects; these connections may be removed in the copy.

The asynchronous gingiva calculation may begin in parallel with the other modifications of the digital model. For example, the asynchronous thread may be processed during one or more user-input processes 1905 (e.g., using a user interface to receive user input, commands and the like). The asynchronous operation may operate in parallel 1907 and may generate a gingiva strip. Optionally the digital model used for generating the gingiva strip may be scanned to generate a CRC code 1909 that may be compared to a CRC code from the 3D model following the subsequent processes 1911. In FIG. 19, the comparison 1913 may compare the models directly or the CRC codes may be compared. If the copy of the 3D digital model is significantly different (e.g., not identical or not identical over the gingival region) as compared to the current 3D digital model from the main thread, then the gingiva strip may be determined from the current 3D digital model 1917, rather than that estimated in parallel from the earlier copy. However, if the copy is substantially the same (e.g., identical, particularly over the gingival region) as the current 3D model, then the 3D digital model may be modified to incorporate the gingiva strip from the copy 1915.

In some examples, in either the asynchronous thread or the main thread, the process of determining the gingiva strip may take a period of time (e.g., 30 seconds) to complete, thus in a number of cases the parallel, synchronous processing described herein may save a significant amount of time. In some examples the asynchronous thread may not finish calculating the gingiva strip before the porting step of the main thread is completed and the result are required. In this case the main thread may wait, e.g., for 10 seconds, and result are not received in this time window, the calculation may be performed in the main thread, and results from asynchronous thread may be ignored. As mentioned, in some cases a user may change the jaw scan (the 3D digital model) before the porting step, but after the copy has been made. In this case, as mentioned above, the asynchronous operation result may not be applied, as the source data were changed. In some cases a scan checksums (e.g., CRC code) may be used; for example a CRC for each jaw may be stored with or immediately after the copy is made and may be compared with a new CRC scan (e.g., a jaw scan checksum) during the porting step. If the results are different, then the gingiva strip may be calculated in the main thread and results from asynchronous thread may be ignored.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like. For example, any of the methods described herein may be performed, at least in part, by an apparatus including one or more processors having a memory storing a non-transitory computer-readable storage medium storing a set of instructions for the processes(s) of the method.

While various embodiments have been described and/or illustrated herein in the context of fully functional computing systems, one or more of these example embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may configure a computing system to perform one or more of the example embodiments disclosed herein.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

In general, any of the apparatuses and methods described herein should be understood to be inclusive, but all or a sub-set of the components and/or steps may alternatively be exclusive and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method, the method comprising:
performing one or more processing steps on a digital model of a patient's dentition;
copying at least a portion of the digital model of the patient's dentition to create a copy the digital model of the patient's dentition;
performing one or more user-input processes on the digital model of the patient's dentition;
generating, in parallel with the performance of the one or more user-input processes, a gingiva strip from the copy of the digital model of the patient's dentition;
comparing the digital model of the patient's dentition to the copy of the digital model of the patient's dentition; and
modifying the digital model of the patient's dentition to include the gingiva strip if the copy of the digital model of the patient's dentition is substantially unchanged from the digital model of the patient's dentition, otherwise generating a second gingiva strip from the digital model of the patient's dentition and modifying the digital model of the patient's dentition to include the second gingiva strip.

2. The method of claim 1, wherein performing one or more processing steps on a digital model of the patient's dentition comprises modifying the digital model of the patient's dentition.

3. The method of claim 1, wherein performing one or more processing steps on a digital model of the patient's dentition comprises modifying the digital model to add or remove features.

4. The method of claim 1, wherein performing one or more processing steps on a digital model of the patient's dentition comprises modifying the digital model to move one or more teeth.

5. The method of claim 1, wherein performing one or more processing steps on a digital model of the patient's dentition comprises one or more of: determining collisions, identifying interproximal reductions, and/or modifying a clinical crown.

6. The method of claim 1, further comprising receiving the digital model of a patient's dentition.

7. The method of claim 1, wherein performing one or more user-input processes on the digital model of the patient's dentition comprises receiving one or more user inputs from a user interface.

8. The method of claim 1, wherein performing one or more user-input processes on the digital model of the patient's dentition comprises one or more of: confirming a tooth axis, reviewing tooth position, modifying one or more settings, and reviewing automatically-generated comments.

9. The method of claim 1, wherein comparing the digital model of the patient's dentition to the copy of the digital model of the patient's dentition comprises comparing a cyclic redundancy check (CRC) code for the digital model of the patient's dentition to a CRC code for the copy of the digital model of the patient's dentition.

10. The method of claim 9, further comprising calculating the CRC code for the copy of the digital model of the patient's dentition while generating the gingiva strip from the copy of the digital model of the patient's dentition.

11. The method of claim 1, further comprising generating a treatment plan from the modified digital model of the patient's dentition.

12. A method, the method comprising:
performing a processing step on a digital model of the patient's dentition, wherein the processing step comprises modifying the digital model of the patient's dentition;
copying a first portion of the digital model of the patient's dentition to create a copy of the patient's dentition;
performing one or more user-input processes on the digital model of the patient's dentition, wherein the user-input processes comprises receiving one or more user inputs from a user interface;
generating, in parallel with the performance of the one or more user-input processes, a gingiva strip from the copy of the digital model of the patient's dentition;
comparing the digital model of the patient's dentition to the copy of the digital model of the patient's dentition by comparing a cyclic redundancy check (CRC) code for the digital model of the patient's dentition to a CRC code for the copy of the digital model of the patient's dentition; and
modifying the digital model of the patient's dentition to include the gingiva strip if the copy of the digital model of the patient's dentition is substantially unchanged from the digital model of the patient's dentition, otherwise generating a second gingiva strip from the digital model of the patient's dentition and modifying the digital model of the patient's dentition to include the second gingiva strip.

13. A non-transitory computer-readable medium comprising one or more computer-executable instructions that, when executed by at least one processor of a computing device, cause the computing device to perform the method of:
performing one or more processing steps on a digital model of a patient's dentition;
copying at least a first portion of the digital model of the patient's dentition to create a copy of the digital model of the patient's dentition;
performing one or more user-input processes on the digital model of the patient's dentition;
generating, in parallel with the performance of the one or more user-input processes, a gingiva strip from the copy of the digital model of the patient's dentition;
comparing the digital model of the patient's dentition to the copy of the digital model of the patient's dentition; and
modifying the digital model of the patient's dentition to include the gingiva strip if the copy of the digital model of the patient's dentition is substantially unchanged from the digital model of the patient's dentition, otherwise generating a second gingiva strip from the digital model of the patient's dentition and modifying the digital model of the patient's dentition to include the second gingiva strip.

14. The non-transitory computer-readable medium of claim 13, wherein performing one or more processing steps on a digital model of the patient's dentition comprises modifying the digital model of the patient's dentition.

15. The non-transitory computer-readable medium of claim 13, wherein performing one or more processing steps on a digital model of the patient's dentition comprises modifying the digital model to add or remove features.

16. The non-transitory computer-readable medium of claim 13, wherein performing one or more processing steps on a digital model of the patient's dentition comprises modifying the digital model to move one or more teeth.

17. The non-transitory computer-readable medium of claim 13, wherein performing one or more processing steps on a digital model of the patient's dentition comprises one or more of: determining collisions, identifying interproximal reductions, and/or modifying a clinical crown.

18. The non-transitory computer-readable medium of claim 13, wherein performing one or more user-input processes on the digital model of the patient's dentition comprises receiving one or more user inputs from a user interface.

19. The non-transitory computer-readable medium of claim 13, wherein performing one or more user-input processes on the digital model of the patient's dentition comprises one or more of: confirming a tooth axis, reviewing tooth position, modifying one or more settings, and reviewing automatically-generated comments.

20. The non-transitory computer-readable medium of claim 13, wherein comparing the digital model of the patient's dentition to the copy of the digital model of the patient's dentition comprises comparing a cyclic redundancy check (CRC) code for the digital model of the patient's dentition to a CRC code for the copy of the digital model of the patient's dentition.

21. The non-transitory computer-readable medium of claim 20, further comprising calculating the CRC code for the copy of the digital model of the patient's dentition while generating the gingiva strip from the copy of the digital model of the patient's dentition.

22. The non-transitory computer-readable medium of claim 13, further comprising generating a treatment plan from the modified digital model of the patient's dentition.

23. A system comprising:
one or more processors;
a memory coupled to the one or more processors, the memory storing computer-program instructions, that, when executed by the one or more processors, perform a computer-implemented method comprising:
performing one or more processing steps on a digital model of a patient's dentition;
copying at least a first portion of the digital model of the patient's dentition to create a copy of the digital model of the patient's dentition;
performing one or more user-input processes on the digital model of the patient's dentition;
generating, in parallel with the performance of the one or more user-input processes, a gingiva strip from the copy of the digital model of the patient's dentition;
comparing the digital model of the patient's dentition to the copy of the digital model of the patient's dentition; and
modifying the digital model of the patient's dentition to include the gingiva strip if the copy of the digital model of the patient's dentition is substantially unchanged from the digital model of the patient's dentition, otherwise generating a second gingiva strip from the digital model of the patient's dentition and modifying the digital model of the patient's dentition to include the second gingiva strip.

24. The system of claim 23, wherein performing one or more processing steps on a digital model of the patient's dentition comprises modifying the digital model of the patient's dentition.

25. The system of claim 23, wherein performing one or more processing steps on a digital model of the patient's dentition comprises modifying the digital model to add or remove features.

26. The system of claim 23, wherein performing one or more processing steps on a digital model of the patient's dentition comprises modifying the digital model to move one or more teeth.

27. The system of claim 23, wherein performing one or more processing steps on a digital model of the patient's dentition comprises one or more of: determining collisions, identifying interproximal reductions, and/or modifying a clinical crown.

28. The system of claim 23, wherein performing one or more user-input processes on the digital model of the patient's dentition comprises receiving one or more user inputs from a user interface.

29. The system of claim 23, wherein performing one or more user-input processes on the digital model of the patient's dentition comprises one or more of: confirming a tooth axis, reviewing tooth position, modifying one or more settings, and reviewing automatically-generated comments.

30. The system of claim 23, wherein comparing the digital model of the patient's dentition to the copy of the digital model of the patient's dentition comprises comparing a cyclic redundancy check (CRC) code for the digital model of the patient's dentition to a CRC code for the copy of the digital model of the patient's dentition.

31. The system of claim 30, further comprising calculating the CRC code for the copy of the digital model of the patient's dentition while generating the gingiva strip from the copy of the digital model of the patient's dentition.

32. The system of claim 23, further comprising generating a treatment plan from the modified digital model of the patient's dentition.
